(19) United States
(12) Patent Application Publication
(10) Pub. No.: US 2020/0407735 A1
(43) Pub. Date: Dec. 31, 2020

(54) VECTOR FOR TRANSFORMATION, TRANSFORMANT, AND TRANSFORMANT-DERIVED PRODUCT

(71) Applicants: NIPPON STEEL TRADING CORPORATION, Minato-ku, Tokyo (JP); NATIONAL UNIVERSITY CORPORATION TOKYO UNIVERSITY OF AGRICULTURE AND TECHNOLOGY, Fuchu-shi, Tokyo (JP)

(72) Inventor: Sakae Suzuki, Tokyo (JP)

(73) Assignees: NIPPON STEEL TRADING CORPORATION, Minato-ku, Tokyo (JP); NATIONAL UNIVERSITY CORPORATION TOKYO UNIVERSITY OF AGRICULTURE AND TECHNOLOGY, Fuchu-shi, Tokyo (JP)

(21) Appl. No.: 16/634,854

(22) PCT Filed: Aug. 1, 2018
(86) PCT No.: PCT/JP2018/028809

§ 371 Date: Jan. 28, 2020

(30) Foreign Application Priority Data
Aug. 1, 2017 (JP) 2017-149274

(51) Int. Cl. C12N 15/82; A01H 5/10; C12N 5/14; A01H 6/60

(52) U.S. Cl. CPC C12N 15/8201; A01H 6/604; C12N 5/14; A01H 5/10

(57) ABSTRACT

A vector includes one or more promoters. The promoters are expressed specifically in a cottonseed surface or a cotton fiber or both of the cottonseed surface and the cotton fiber.

### CROSS-REFERENCE TO RELATED APPLICATIONS

This application is based upon and claims the benefit of priority from Japanese Patent Application No. 2017-149274, filed on Aug. 1, 2017. Japanese Patent Application No. 2018-144788, filed on Aug. 1, 2018, is also based upon and claims the benefit of priority from Japanese Patent Application No. 2017-149274, filed on Aug. 1, 2017.

### TECHNICAL FIELD

The present invention relates to vectors for transforming plants, transformants, and transformant-derived products. More particularly, the present invention relates to vectors for transforming cotton, transformants, and transformant-derived products.

### BACKGROUND ART

Heretofore, colored cotton has had only two colors, namely brown and green, and has been unsuitable for commercial processing due to its lesser fiber length, strength, yield, spinning properties, and the like, and therefore there has been a demand to make high-quality cotton of multiple colors. Also, although colored cotton at present is known to have an accumulation of flavonoids in its fiber part, there is no example of introducing these traits into commercial varieties by cross-breeding. Methods for cotton transformation were already developed in the 1980s, but multicoloring has not yet seen success.

Meanwhile, technology for changing the color of flowers and leaves by cloning and introducing pigment synthesis genes such as flavonoid, carotenoid, betalain, and the like has been developed based on genetic modification technology, but has not yet elevated to a level where the color of cotton can be changed freely by modifying genes. So, there has been a demand to develop vectors and/or transformation methods that are suitable for making cotton fiber develop colors.

### BRIEF DESCRIPTION OF DRAWINGS

FIG. 1 is a diagram illustrating a biosynthetic pathway of flavonoids;

FIG. 2 is a plasmid map of a vector pRI-GhRDL1p-Atpap1/35Sp-GhHOX3 used in the present invention;

FIG. 3 is a plasmid map of a vector pRI-GhEXPAp-Atpap1/35Sp-GhHOX3 used in the present invention;

FIG. 4 is a plasmid map of a vector pRI-GhRDL1p-Atpap1/GhEXPAp-Atpap1/35Sp-GhHOX3 used in the present invention;

FIG. 5 shows SEQ ID NO: 14, the base sequence of a GhRDL1 promotor region;

FIG. 6 shows SEQ ID NO: 15, the base sequence of the GhEXPA promotor region;

FIG. 7 is a plasmid map of a vector pRI-35Sp-Atpap1;

FIG. 8 shows photographs of pigment expressions by various plasmids in cotton fiber;

FIG. 9 shows a photograph, in which only the trichome cells of leaves of transgenic tobacco, obtained by using pRI-GhRDL1p-Atpap1/GhEXPAp-Atpap1/35Sp-GhHOX3, are colored red;

FIG. 10 shows a photograph, in which the trichome cells of young leaves of transgenic tobacco, obtained by using pRI-GhRDL1p-Atpap1/GhEXPAp-Atpap1/35Sp-GhHOX3, are colored distinctively red;

FIG. 11 shows a photograph, in which the petals of transgenic tobacco, obtained by using pRI-GhRDL1p-Atpap1/GhEXPAp-Atpap1/35Sp-GhHOX3, are colored red;

FIG. 12 shows a photograph, in which the trichome cells of leaves of transgenic tobacco, obtained by using pRI-GhRDL1p-Atpap1/GhEXPAp-Atpap1, are colored redder; and

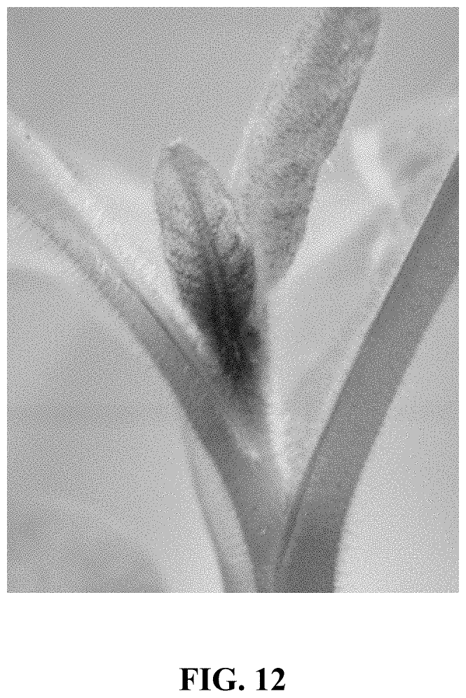

FIG. 13 shows a photograph, in which the base part of petals of transgenic tobacco, obtained by using pRI-GhRDL1p-Atpap1/GhEXPAp-Atpap1, is colored red, and an enlarged photograph thereof.

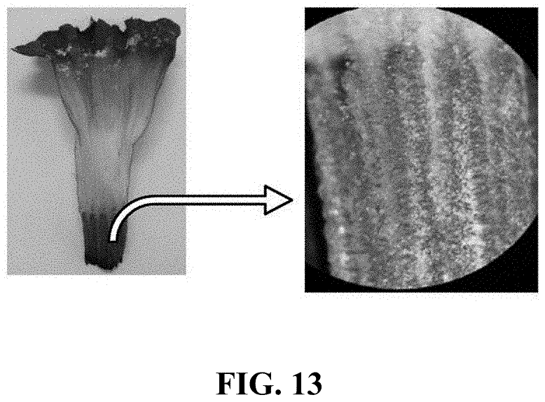

### DETAILED DESCRIPTION

Vectors containing promoters and transcription factors expressed specifically in plants, particularly cotton fiber tissues, cotton transformed with them, and products derived from transformed cotton, are provided.

According to the present invention, the following inventions are provided.

(1) A vector including at least one or more promoters that are expressed specifically in a cottonseed surface and/or a cotton fiber, or a vector including at least one or more promoters that are expressed specifically in a cottonseed surface and/or a cotton fiber, and at least one transacting factor gene that is functionally linked to the promoters and activates transcription from the promoters.

(2) In the vector according to (1), the promoters that are expressed specifically in the cottonseed surface and/or the cotton fiber is one of:

(i) a GhRDL1 and/or GhEXPA promoter;

(ii) a promoter that hybridizes with the GhRDL1 or GhEXPA promoter under a stringent condition, and that is expressed specifically in the cottonseed surface and/or the cotton fiber; and

(iii) a promoter that has 70% or higher homology with the GhRDL1 or the GhEXPA promoter, and that is expressed specifically in the cottonseed surface and/or the cotton fiber.

(3) In the vector according to (1) or (2), the transacting factor gene is one of:

(i) a DNA that encodes a GhHOX3 protein;

(ii) a DNA that encodes a protein including 60% or higher amino acid identity with the GhHOX3 protein and including a transcriptional activation function equivalent to the GhHOX3 protein;

(iii) a DNA that encodes a protein including 70% or higher homology with the DNA of (i) and including a transcriptional activation function equivalent to the GhHOX3 protein; and

(iv) a DNA that encodes a protein hybridizing with the DNA of (i) under a stringent condition and including a transcriptional activation function equivalent to the GhHOX3 protein.

(4) In the vector according to one of (1) to (3), the vector includes at least one transcription factor gene and/or a pigment biosynthetic gene that are functionally linked and promote expression of a pigment biosynthetic gene group.

(5) In the vector according to (4), the transcription factor gene promotes the expression of the pigment biosynthetic gene group is one of:

(i) a DNA that encodes an Atpap1 protein;

(ii) a DNA that encodes a protein including 60% or higher amino acid identity with the Atpap1 protein and including a transcriptional activation function equivalent to the Atpap1 protein;

(iii) a DNA that encodes a protein including 70% or higher homology with the DNA of (i) and including a transcriptional activation function equivalent to the Atpap1 protein; and

(iv) a DNA that encodes a protein hybridizing with the DNA of (i) under a stringent condition and including a transcriptional activation function equivalent to the Atpap1 protein.

(6) A vector including a sequence of one of SEQ. ID NOs. 1 to 3. More preferably, a vector that consists of the sequence one of SEQ. ID NOs. 1 to 3.

(7) The vector according to one of (1) to (6), further including a border region of a T-DNA.

(8) An agrobacterium, in which the vector according to (7) is transformed.

(9) A cell, a tissue, a callus, a transgenic plant, and/or a seed thereof, transformed with the agrobacterium according to (8).

(10) A clonal plant, a progeny plant, and/or a seed thereof, derived from the transgenic plant or the seed thereof according to (9).

(11) In the cell, the tissue, the callus, the transgenic plant and/or the seed thereof, and the clonal plant, the progeny plant, and the seed thereof according to (9) or (10), the transformed cell, tissue, and callus, the transgenic plant, and/or the seed thereof, and the clonal plant, the progeny plant, and the seed thereof are derived from cotton.

(12) A method for producing the vector according to (4), (6) or (7), and/or the cotton cell, the tissue, the callus, and the transgenic plant and/or the seed thereof, transformed with the agrobacterium according to (8), and the clonal plants, progeny plants, and the seed thereof.

(13) A cotton fiber, a fabric, or clothing derived from the transgenic plant according to (11) or (12).

(14) A cotton fiber, a fabric, or clothing including a foreign flower color-related gene.

According to the present invention, genes can be efficiently expressed in cotton plants.

The present invention provides vectors that allow tissue-specific expression of plants, especially cotton. With the present invention, cotton preferably refers to, but is not limited to, the following species that are included in gossypium and grown for commercial purposes: Gossypium hirsutum, Gossypium barbadense, Gossypium arboretum, and Gossypium herbaceum.

The vectors of the present invention include at least one or more promoters that are expressed specifically in cottonseed surfaces and/or cotton fibers. For these promoters, for example, cottonseed surface-specific and/or cotton fiber-specific promoters such as GhRDL1, GhEXPA, GhCesA4, GhACT1 and GhDET2 are suitable for use, but these are by no means limiting. That is, any promoters that can be expressed specifically in cottonseed surfaces and/or cotton fibers may be used. These promoters preferably have cis-elements (cis-factors) for allowing cottonseed surface-specific and/or cotton fiber-specific expression, but may be controlled differently and expressed specifically in cottonseed surfaces and/or cotton fibers.

Furthermore, promoters that are expressed constitutively, and that are also expressed in cottonseed surfaces and/or cotton fibers can be used as well. In this case, a system to inhibit the expression of target genes in tissues where the expression is not needed, may be used. For example, the expression of target genes in tissues where the target genes are unneeded for expression can be inhibited by having RNAi or antisense RNA expressed specifically in these tissues.

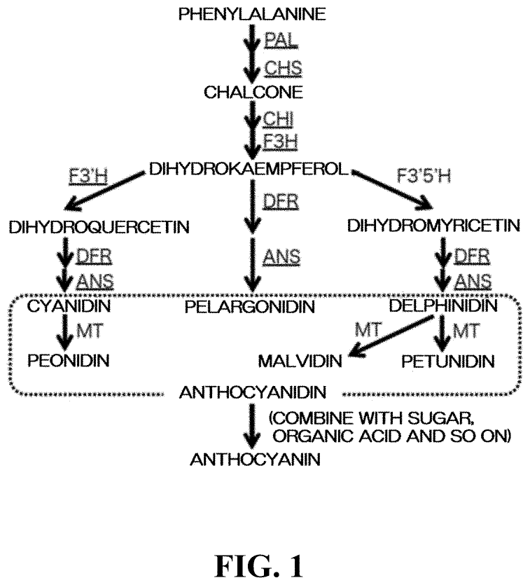

The genes to be functionally linked to the downstream of these promoters and to be expressed in target tissues are not particularly limited, but for example, gene groups that relate to the production of pigments or transcription factors that control these gene groups are preferable. Gene groups that relate to the production of pigments might include gene groups that relate to flavonoid pigment biosynthesis, gene groups that relate to carotenoid pigment biosynthesis, gene groups that relate to betalain pigment biosynthesis, and so forth, but these are not limiting, and any genes relating to the production of pigments may be used. The origin of pigment biosynthetic gene groups may be plants, animals, microorganisms, and the like, and is not particularly limited. Transcription factors to control the pigment biosynthetic gene groups might include, for example, the Atpap1 genes of Arabidopsis thaliana, which activate the expression of flavonoid biosynthesis gene groups (activate the genes underlined in FIG. 1), but these are not limiting. For example, a transcription factor that has an MYB, bHLH, and WDR domain and controls pigment biosynthesis may be used.

The vectors of the present invention may further contain transacting factors that activate the above cis-elements. These transacting factors might include, for example, the GhHOX3, GhMYB109, GhMYB25, GhMYB2A and GhMYB2D genes of cotton, but these are not limiting. These transacting factors can promote the expression of genes by binding with the above cis-elements and promoting transcription from the above promoters. The promoters to be bound to the upstream of genes that encode the transacting factors may be promoters that are expressed constitutively or may be promoters that are tissue-specifically and/or stage-specifically expressed, but it is also preferable to use promoters that are at least expressed in tissues where the target genes are wanted to be expressed.

The vectors of the present invention may further contain genes that are involved in the biosynthesis of pigments. The genes to be involved in pigment biosynthesis include, for example, flavonoid pigment biosynthetic genes, carotenoid pigment biosynthetic genes, and betalain biosynthesis genes, but these are not limiting, and any genes that are involved in pigment-producing biosynthetic pathways can be included in the vectors of the present invention.

Examples of flavonoid pigment biosynthetic genes include, for example, flavonoid 3′,5′-dehydrogenase, which is involved in blue pigment biosynthesis. By introducing and expressing this enzyme gene in plants having an anthocyan synthesis pathway and not having flavonoid 3′,5′-dehydrogenase, the color can be shifted towards purple to blue. Furthermore, by introducing genes that biosynthesize sugar chains that stabilize delphinidin and genes that add these sugar chains to delphinidin together, delphinidin can be stabilized, and purple to blue can be stabilized. Also, in order to bring the color closer to blue, it is desirable to inhibit the expression of DFR genes by RNAi, antisense method, and so on.

In addition, yellow-pigment biosynthetic genes include genes such as aurone synthase, rutin synthase, carotenoid synthase, and the like.

As for the DNA of the promoter region, the transcription factor genes and the pigment biosynthetic genes, it is possible to use DNA sequences that are naturally found, but it is also possible to use DNA sequences that are partially-mutated (added, deleted, substituted, etc.) as long as these DNA sequences have necessary functions.

For example, a promoter that is hybridized to the DNA of SEQ. ID NO. 14 or 15 under stringent conditions, and that is expressed in a cotton fiber-specific manner may be used as a cotton fiber-specific promoter.

Here, the stringent conditions may be low stringent conditions, medium stringent conditions or high stringent conditions. The “low stringent conditions” refer to, for example, 5×SSC, 5×Denhardt's solution, 0.5% SDS, 50% formamide, and 32° C. Here, 1×SSC is 150 mM NaCl and 15 mM sodium citrate and pH 7.0, and 5×Denhardt's solution is 0.1% (w/v) BSA, 0.1% (w/v) Ficol (registered trademark) 400, and 0.1% (w/v) polyvinyl pyrrolidone (PVP). Also, the “medium stringent conditions” refer to, for example, 50° C., 2×SSC, and 0.1% SDS. The “high stringent conditions” refer to, for example, 65° C., 0.1×SSC and 0.1% SDS. Under these conditions, it is expected that DNA having higher homology can be obtained efficiently as the temperature is increased. However, there may be multiple factors that influence the stringency of hybridization such as temperature, probe concentration, probe length, ionic strength, time, and salt concentration, and those skilled in the art can achieve the same stringency by appropriately selecting these elements.

Whether a DNA that is hybridized and obtained from a library by plaque hybridization or colony hybridization under these stringent conditions has cotton fiber-specific promoter activity can be confirmed easily by linking it.

Furthermore, a DNA that has 99%, 98%, 97%, 96%, 95%, 94%, 93%, 92%, 91%, 90%, 85%, 80%, 75%, 70%, 65%, 60% or higher DNA sequence homology with the cotton fiber-specific promoter of SEQ. ID NO. 14 or 15, and that shows cotton fiber-specific expression may be also used as a promoter of the present invention. In this case, the upper limit of homology is 100%. DNA homology can be determined by programs well known to those skilled in the art, such as NCBI BLAST (registered trademark). The DNA sequence homology and amino acid sequence identity as used herein refer to the homology and identity in the standard setting of NCBI BLAST.

Also, for the transcription factor or pigment biosynthetic gene, a gene that has 99% or higher, 98% or higher, 97% or higher, 96% or higher, 95% or higher, 94% or higher, 93% or higher, 92% or higher, 91% or higher, 90% or higher, 85% or higher, 80% or higher, 75% or higher, 70% or higher, 65% or higher, or 60% or higher DNA homology, and that has the transcriptional activation ability or pigment synthesis activity equivalent to that of the original gene can be linked to the vectors of the present invention and used. In this case, the upper limit of identity is 100%. Here, “equivalent” simply means the same kind, and the strength of activity does not necessarily have to be the same. These homologous genes can be obtained by screening a cDNA library or a genomic library based on plaque hybridization, colony hybridization, and the like, and the transcription factor activity and pigment biosynthesis activity can also be confirmed by combining techniques well known to those skilled in the art.

Furthermore, the transcription factor or the pigment biosynthetic gene may be a DNA that has 99% or higher, 98% or higher, 97% or higher, 96% or higher, 95% or higher, 94% or higher, 93% or higher, 92% or higher, 91% or higher, 90% or higher, 85% or higher, 80% or higher, 75% or higher, 70% or higher, 65% or higher, or 60% or higher identify with the protein the DNA encodes, and that encodes a protein with a transcriptional activation ability or pigment synthesis activity equivalent to that of the original gene. Here, “equivalent” simply means the same kind, and the strength of activity does not necessarily have to be the same.

It is possible to have pigments expressed in cotton fibers by transforming a plant with a vector containing a transcription factor (for example, Atpap1) and a transacting factor of a biosynthetic pathway linked to a promoter of the present invention, especially by having the promoters expressed specifically in cottonseed surfaces and/or cotton fibers. However, in this case, the pigment biosynthetic gene group to match the transcription factor needs to be present. For example, in the event Atpap1 is used, a gene group that is capable of developing at least one color of flavonoid pigment biosynthetic gene needs to be present. However, it is not necessary to have all the biosynthetic genes of cyanidin, pelargonidin and delphinidin. Alternatively, it is possible to biosynthesize pigments by introducing all the pigment synthesis system gene groups.

The vectors of the present invention preferably contain either the border-region DNAs at both ends of the T-DNA (the right border region (Rb) and the left border region (Lb)) of agrobacterium, or Rb.

To introduce a gene into a plant, the Ti plasmid system or the Ri plasmid system of agrobacterium can be used. By using an intermediate vector method, in which a T-DNA region is substituted in a Ti plasmid by double crossover recombination, and by introducing this recombined Ti plasmid in agrobacterium and infecting the plant cells, a T-DNA region can be inserted in the nuclear genome of the plant cells.

The vectors of the present invention are preferably a Ti-plasmid binary vector system. In the event binary vectors are used, T-DNA and the gene group that is needed to introduce the T-DNA region into plants are contained in different plasmids. In this case, agrobacterium containing a plasmid (for example, LBA4404 or the like) with a gene group having a function for introducing a T-DNA region into plants in advance is transformed with a plasmid containing T-DNA. As for the method of transformation, the tri-parental mating method, the electroporation method and the like can be used suitably.

Agrobacterium transformed (or having been subjected to double crossover recombination) with a plasmid containing T-DNA is liquid-cultured in LB medium and the like, and then brought into contact with plant tissue fragments and co-cultured. If necessary, acetosyringone may be added. For the method of bringing agrobacterium into contact with plant tissue fragments, the vacuum infiltration method, the dip method and the like are suitable for use. While nurse culture (feeder culture) is preferable for the co-culture after the contact, with many plants, transformants can be obtained without feeder culture. The period of co-culture is preferably approximately two days to one week, but this is not limiting. That is, the co-culture has only to be kept for a range of period so that problems such as the overgrowth of agrobacterium killing the plant tissue fragments, the amount of infection being so low and resulting in an inability to have a sufficient number of transformed calluses, and so on, do not arise.

For the plant tissue fragments, leaf pieces, stems, hypocotyls, embryos, shoot apices, roots, calluses and the like are suitable for use, but these are not limiting.

The co-cultured plant tissue fragments are further cultured in a medium containing antibiotics such as carbenicillin, to remove agrobacterium.

The transformed calluses, derived from the plant tissues where agrobacterium has been removed, are then re-differentiated by normal tissue culture techniques, and normal plants can be obtained by rooting and acclimatization.

If the plant of the tissue fragments used is fixed, a progeny plant can be obtained by taking the seeds. Plants derived from F1 seeds may be vegetatively grown (by cutting-propagation and the like) and propagated as clonal plants, or may be fixed as varieties by repeating backcrossing. In addition, embryos may be derived from transformants to prepare artificial seeds.

From the cottonseeds obtained as described above, cotton fibers can be prepared into yarns and fabrics by methods well known to those skilled in the art, and, furthermore, clothing can be produced. That is, final products can be manufactured through the process of picking cotton crops (cotton balls), separating cotton fibers and seeds in a cotton gin factory, spinning them into yarns, weaving, dyeing, textile finishing, and sewing.

The method of manufacturing fabrics and products will be described below in more detail. At the same time compressed cotton, which is the raw material, is unraveled, the leaf pieces, seed pieces, sand dust and the like contained in the raw material are removed, and the raw material is formed into a sheet. Next, the fibers are separated one by one by combing the sheet-like wrap, and small dust and short fibers are removed by making them parallel. The remaining long fibers are made substantially parallel, bundled, and formed into a string-like sliver. Doubling and drafting of the sliver are repeated several times to make it thin and uniform. Yarn is twisted and spinned by stretching the then-thin sliver up to yarn's thickness, thereby producing a piece of yarn called “single yarn”. Furthermore, two-fold yarn can be made by twisting two of these single yarns in opposite directions. Then, if necessary, the yarn is rolled back into the form of cheese or cone. Using the finished yarn for warp yarn and weft yarn, pieces of cloth for use as fabrics can be woven with a loom. Using the woven fabrics obtained then, clothing, bags and the like can be manufactured by methods well known to those skilled in the art.

Examples of yarns, fabrics, and clothing manufactured from the transformed cotton of the present invention include cotton fabrics and clothing made from cotton fabrics. Examples of cotton fabrics include, but are not limited to, lone, broad, sheeting, CB poplin, oxford, drill and cotton-linen canvas, and canvas. Examples of clothing include, but are not limited to, underwear, shirts, blouses, pants, skirts, T-shirts, cardigans, and tunics. In short, any woven fabrics, knitted fabrics or clothing manufactured from cotton fibers can be manufactured from the cotton of the present invention.

For cotton fibers, yarns, fabrics or clothing obtained from seeds containing the genes of the present invention, DNAs may be extracted using the regular method, and the presence of foreign genes may be confirmed by the PCR method. As for the method of extracting DNAs from cotton fibers, yarns, fabrics and clothing, DNAs may be extracted using a DNA extraction kit that is commercially available, or may be extracted using the CTAB method and the like (see, for example, U.S. Pat. No. 9,938,586, WO2010/056642, etc.).

### Embodiment

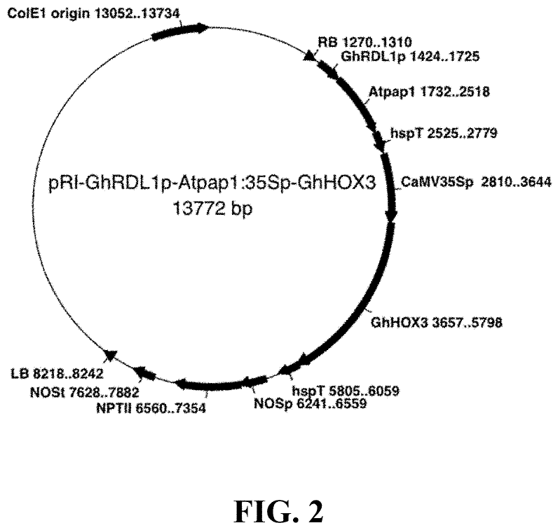

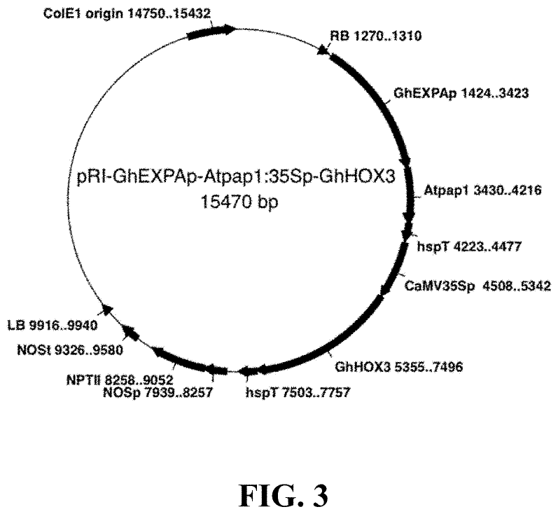

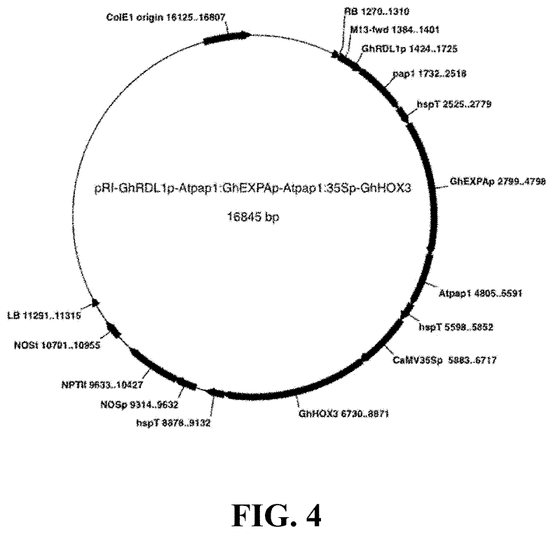

1. Method for Preparing Binary Vectors pRI-GhRDL1p-Atpap1/35Sp-GhHOX3 (FIG. 2, SEQ. ID NO. 1) pRI-GhEXPAp-Atpap1/35Sp-GhHOX3 (FIG. 3, SEQ. ID NO. 2) pRI-GhRDL1p-Atpap1/GhEXPAp-Atpap1/35Sp-GhHOX3 (FIG. 4, SEQ. ID NO. 3)

The following primer sequences are used.

The Primer Sequences:
(SEQ. ID NO. 4)
AtpapI U-XbaI:
CCAGTGTCTAGACTATCTTTGTTCCATGGAGGG
(SEQ. ID NO. 5)
AtpapI L-SacI:
CCAGTGGAGCTCCACAAACGCAAACAAATGTTC
(SEQ. ID NO. 6)
GhRDL1p U-HindIII:
CCAGTGAAGCTTAATTAGTTATGTTTGGTAAAT
(SEQ. ID NO. 7)
GhRDL1p L-XbaI:
CCAGTGTCTAGACTAGAACAGGAGTGACTAATT
(SEQ. ID NO. 8)
GhEXPAp U-HindIII:
CCAGTGAAGCTTTTTAAGCAAAAAATTAATAGT
(SEQ. ID NO. 9)
GhEXPAp L-XbaI:
CCAGTGTCTAGATTGAGTAAGAGCTAGCTAGCT
(SEQ. ID NO. 10)
GhHOX3 U-XbaI:
CCAGTGTCTAGAATGGATTGCGGAAGCGGCGGC
(SEQ. ID NO. 11)
GhHOX L-SacI:
CCAGTGGAGCTCTCAAGAACTAGGACAATTCAA
(SEQ. ID NO. 12)
hspT L-PstI:
ACTACTCTGCAGAATTCCTTATCTTTAATCATA
(SEQ. ID NO. 13)
GhRDL1p U-SphI:
CCAGTGGCATGCAATTAGTTATGTTTGGTAAAT

### The Base Sequence of the Promoter Region:

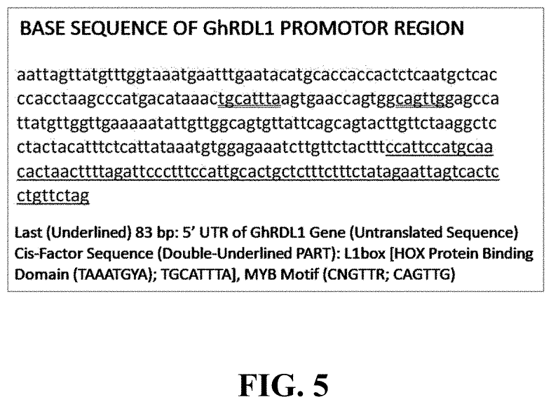

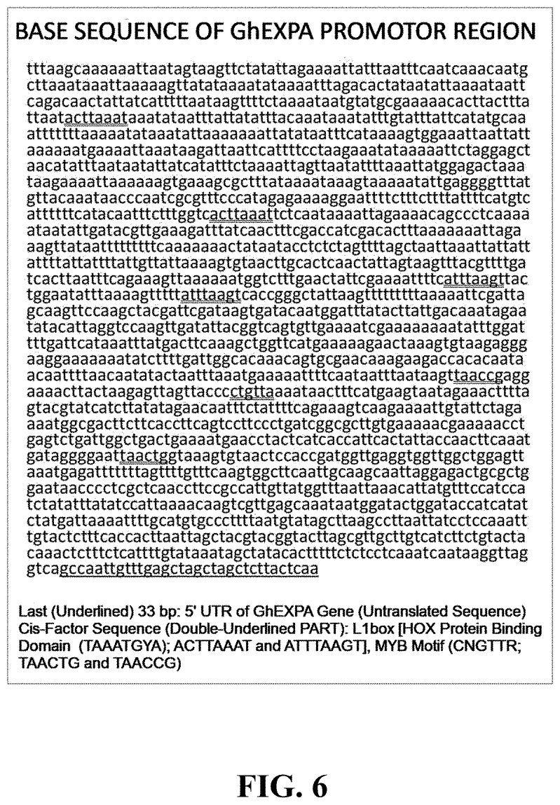

The base sequences and cis factors of the GhRDL1 promoter region (SEQ. ID NO. 14) and the GhEXPA promoter region (SEQ. ID NO. 15) are shown in FIG. 5 and FIG. 6. In addition, the base sequences of the regions encoding the proteins of Atapa1 (accession No. AK221639) and GhHOX3 (accession No. KJ595847) are shown as SEQ. ID NOs. 16 and 17.

Preparation of pRI-GhRDL1p-Atpap1, pRI-GhEXPAp-Atpap1 and 35Sp-GhHOX3:

From the cDNA of Arabidopsis thaliana, the Atpap1 gene (787 bp, anthocyanin biosynthesis transcription factor) was amplified by the PCR method, using the primers Atpap1 U-XbaI and Atpap1 L-SacI. From the genomic DNA of cotton (Gossypium hirsutum), GhRDL1p (302 bp) and GhEXPAp (2000 bp) of cotton fiber-specific promoter sequences, and GhHOX3 gene (2142 bp) of a cotton fiber-specific transcription factor were amplified by the PCR method, using the following primers: GhRDL1p U-HindIII and GhRDL1p L-XbaI, GhEXPAp U-HindIII and GhEXPAp L-XbaI, and GhHOX3 U-XbaI and GhHOX L-SacI.

These isolated genes were introduced into the basic vector pRI201-AN-GUS (TaKaRa). pRI-35Sp-Atpap1 was prepared by inserting an insert sequence of an XbaI-Atpap1-SacI fragment in the pRI201-AN-GUS vector, from which the GUS gene had been removed by using restriction enzymes XbaI and SacI. Furthermore, the CaMV35S promoter gene of pRI-35Sp-Atpap1 was removed by using restriction enzymes HindIII and XbaI, and inserts of a HindIII-GhRDL1p-XbaI fragment or a HindIII-GhEXPAp-XbaI fragment were inserted as promoter sequences, and pRI-GhRDL1p-Atpap1 and pRI-GhEXPAp-Atpap1 were prepared.

pRI-35Sp-GhHOX3 was prepared by inserting an insert sequence of an XbaI-GhHOX3-SacI fragment in the pRI201-AN-GUS vector, from which the GUS gene had been removed by restriction enzymes XbaI and SacI.

Preparation of pRI-GhRDL1p-Atpap1/35Sp-GhHOX3 and pRI-GhEXPAp-Atpap1/35Sp-GhHOX3:

Using the following PCR primers, GhRDL1p U-HindIII and hspT L-PstI, and GhEXPAp U-HindIII and hspT L-PstI, HindIII-[pRI-GhRDL1p-Atpap1]-PstI fragment was amplified from pRI-GhRDL1p-Atpap1, and HindIII-[GhEXPAp-Atpap1]-PstI fragment was amplified from pRI-GhEXPAp-Atpap1. Each insert fragment was inserted in the pRI-35Sp-GhHOX3 vector, which had been cleaved with restriction enzymes HindIII and PstI, to prepare pRI-GhRDL1p-Atpap1/pp-GhHOX3 and pRI-GhEXPAp-Atpap1/35Sp-GhHOX3.

Preparation of pRI-GhRDL1p-Atpap1/GhEXPAp-Atpap1/35Sp-GhHOX3: Using the following PCR primers, GhRDL1p U-SphI and hspT L-PstI, SphI-[pRI-GhRDL1p-Atpap1]-PstI fragment was amplified from pRI-GhRDL1p-Atpap1. This fragment was inserted into the pRI-GhEXPAp-Atpap1/35Sp-GhHOX3 vector, which had been cleaved with restriction enzymes SphI and PstI, as an insert, to prepare pRI-GhRDL1p-Atpap1/GhEXPAp-Atpap1/35 Sp-GhHOX3.

PCR reaction, agarose gel electrophoresis, collection and refining of target fragments from agarose gel were conducted according to a manual, by using TaKaRa Ex Taq, and NucleoSpin Gel and PCR Clean-up (TaKaRa). Ligation reaction, transformation into E. coli, and plasmid extraction were conducted according to a manual, by using DNA Ligation Kit Long (TaKaRa), E. coli DH5a Competent Cells (TaKaRa), and NucleoSpin Plasmid EasyPure (TaKaRa).

Introduction of Binary Plasmid into Agrobacterium:

50 μL of Agrobacterium (Agrobacterium tumefaciens) EHA105 or LBA4404 with disarmed Ti plasmid and 2 μL of a plasmid solution were mixed in a cuvette, and the mixture was electroporated under the conditions of 2.5 KV, 125 pFD, and 200 S2, using Gene Pulser GENEPULSER II (BIORAD). The agrobacterium solution after the electroporation was moved to a 500-μL SOC liquid medium in a 1.5 ml tube, and cultured for 1 hour at 28° C. This culture solution was spread on a YEP medium plate, which was adjusted to contain 30 ppm of kanamycin, by using a bacteria spreader. The plate was sealed with Parafilm and incubated overnight at 28° C., and colony formation was confirmed the next day. Introduction of binary plasmids was confirmed by amplifying the target gene sequence by the colony PCR method.

2. Method of Transforming Cotton:

Preparation of Inoculum:

Cotton (Gossypium hirsutum)-seeds were placed in a 1.5-ml eppendorf tube and immersed in 80% ethanol for 30 seconds for surface sterilization, and, after ethanol was discarded and evaporated, and antiformin with an effective chlorine concentration of 1%, supplemented with a small amount of tween 20, was added, the seeds were sterilized for 10 minutes while being inverted and mixed. Antiformin was removed in a clean bench, and the residue was cleansed 10 times with sterile water. The sterilized seeds were sown in MS medium and cultured. As for the conditions of culture, a 90 mm×20 mm sterilized petri dish was used, and culture was carried out at 25° C. in the dark.

Preparation of Agrobacterium Suspension:

After agrobacterium, in which each vector had been introduced and which had been cryopreserved in a glycerol stock, was thawed, 10 mL of YEP medium, supplemented with 30 ppm kanamycin, was added, and the resulting mixture was shaken for 24 hours at 28° C. for selection and proliferation. After that, the mixture was centrifuged at 3000 rpm for ten minutes, and the supernatant was discarded. 10 mL of YEP medium supplemented with 10 ppm acetosyringone was added to the precipitated fungus body and resuspended, to prepare an agrobacterium suspension.

Inoculation and Co-Culture of Agrobacterium:

A seedling hypocotyl was prepared by culturing a seed that had been sown aseptically at 25° C., in the dark for three days. In a clean bench, the seedling was placed in a 90 mm×20 mm sterilized petri dish where filter paper was placed, and the agrobacterium suspension that had been prepared was poured in there. The seedling hypocotyl was soaked in the agrobacterium suspension and cut to 2 to 3 mm, and then inoculated with agrobacterium. Following that, the excess agrobacterium suspension adhered to the hypocotyl was blotted with the filter paper, and the hypocotyl was placed on co-culture medium (1), and cultured. As for the conditions of culture, a petri dish was used, and the culture was carried out for three days at 25° C. in the dark.

Regeneration of Plant:

Following the co-culture, the hypocotyl was cleansed three times with sterile water and transplanted to callus induction selection medium (2) for sterilization of agrobacterium and selection of transformants Subculture was carried out every five to seven days. When the induced callus grew large enough (2 months or more after the inoculation), the callus was transplanted to adventitious bud induction selection medium (3). Adventitious buds, which had grown from the callus and which were approximately 1 to 2 cm, were cut from the callus, and transplanted to adventitious root induction medium (4) to stimulate the growth of roots. The adventitious buds were cultured under continuous illumination, at 25° C., until they expanded long enough. As for the conditions of culture from the induction of callus to the development of adventitious buds, a 90 mm×20 mm sterilized petri dish was used at all times, under continuous illumination, at 25° C. Only the adventitious root induction medium was cultured in a plant box (72×72×100 mm).

Acclimation of Plant:

Individuals with sufficiently expanded adventitious roots were taken out of the medium, and, after the roots were washed with running water, transplanted in a pot, which was 90 mm in diameter, by using sterilized garden plant soil. To keep the humidity, the pot was wrapped in a plastic bag and placed in a phytotron at 25° C. (14 h/10 h day length) for growth. Following this, the plastic bag was removed little by little, for acclimatization.

Confirmation of Transformant:

DNA was extracted from the leaves of the resulting plant body, and part of a kanamycin-resistant gene (nptII gene) was amplified by the PCR method to confirm the transformation.

(1) Co-culture medium: MS medium (Murashige and Skoog medium)+0.1 ppm NAA (1-Naphthaleneacetic acid)+0.1 ppm BAP (6-Benzylaminopurine)

(2) Callus induction selection medium: MS medium+0.1 ppm NAA+0.1 ppm BAP+75 ppm kanamycin+10 ppm meropen (Meropenem Hydrate)

(3) Adventitious bud induction selection medium: MS medium+1 ppm GA3 (Gibberellin)+75 ppm kanamycin+10 ppm meropen

(4) Adventitious root induction medium: ½ MS medium+0.3 ppm IBA (indole-3-acetic acid)+10 ppm meropen

(Transient Expression of Genes in Cotton Fiber Cells)

Preparation of pRI-35Sp-Atpap1:

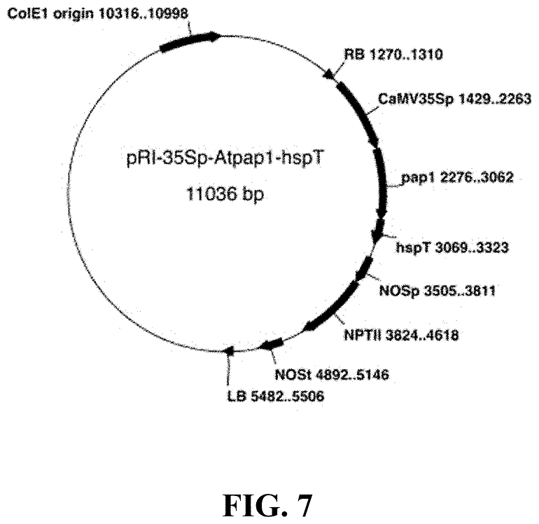

From the cDNA of Arabidopsis thaliana, the Atpap1 gene (787 bp, anthocyanin biosynthesis transcription factor) was amplified based on the PCR method, using the primers Atpap1 U-XbaI and Atpap1 L-SacI. pRI-35Sp-Atpap1 was prepared by inserting an insert sequence of an XbaI-Atpap1-SacI fragment into the pRI201-AN-GUS (TaKaRa) vector, from which the GUS gene had been removed using restriction enzymes XbaI and SacI (FIG. 7).

Transient Expression of Atpap1 in Immature Cottonseeds by Particle Gun Method (Gene Gun)

A particle gun-based gene transfer device (PDS-1000/He, Bio-Rad) was used to introduce the target gene and to confirm transient expression.

Pre-Treatment of Gold Particles:

12 mg of gold particles (1 μm in diameter) was weighed, added 200 μL of 100% ethanol, and vortexed for five minutes. The resulting gold particles were left to stand at room temperature for approximately five minutes, and then centrifuged at 5000 rpm. The supernatant was discarded, 250 μL of 75% ethanol was added, and the resulting gold particles were flicked hard with fingers and mixed well, vortexed for three minutes, then left to stand for approximately five minutes, and centrifuged at 5000 rpm, and the supernatant was discarded. Following this, 300 μL of sterile water was added, and the resulting gold particles were flicked hard with fingers and mixed well, vortexed for one minute, left to stand for one minute, centrifuged at 5000 rpm, and the supernatant was discarded. This process was repeated three times. 200 μL of 50% glycerol was added, and the resulting gold particles were vortexed and mixed.

Coating of Gold Particles with Plasmid DNA:

Each plasmid was adjusted to 1 μg/μL in advance, 5 μg of plasmids was put in a 1.5 mL tube, added 50 μL of 60 mg/mL metal particles prepared as described above, and mixed by pipetting. The mixture was vortexed with the lid open, and, after it was confirmed that the mixture was well mixed, 50 μL of 2.5 M CaCl2 was added, and, furthermore, 10 μL of 0.1 M spermidine was quickly added, the lid was closed, and the mixture was vortexed for three minutes, and left to stand for one minute. Following this, the resulting mixture was centrifuged at 5000 rpm, and the supernatant was discarded. 140 μL of 70% ethanol was added slowly so as not to disturb the precipitation of the gold particles, and was soon sucked out and discarded. In the same manner, the gold particles were cleansed twice with 100% ethanol, added 80 μL of 100% ethanol, and the metal fine particles were made to float by flicking the tube hard, and coated.

Implantation Conditions for Particle Gun:

Inside the clean bench, a macro carrier was placed on a paper towel, 12 μL of ethanol solution of plasmid-coated gold particles was placed in the center of the macro carrier and dried by air. In one implantation, 0.45 mg of gold particles and 750 ng of plasmid DNA were used. After drying, the macro carrier and a stopping screen were set in the device. The gas pressure for implantation was 900 psi, and a rupture disk for 900 psi was used. The distance from the stopping screen to the target immature cottonseed was 6 cm.

Pre-Treatment of Immature Cottonseed for Implantation:

A cotton ovary organ, approximately 10 mm long and containing immature seeds, was cut longitudinally with a scalpel, and fixed to clay so that the cut surface was up. The cut surface shows five to eight immature seeds, approximately 2 mm long, and, in this state, the length of fiber cells on the immature seed surfaces is 0.5 mm or less. The fixed sample was set on the stage of the device body, and implantation was carried out.

Result of Transient Expression of Atpap1 in Immature Cottonseeds:

After the implantation treatment, the immature seeds, placed on a Kimwipes (Registered Trademark) that was sufficiently moistened with sterile water, was placed straight in a plastic petri dish, and placed under a low light condition at 25° C. After the treatment, the state of the immature seeds was observed every other day.

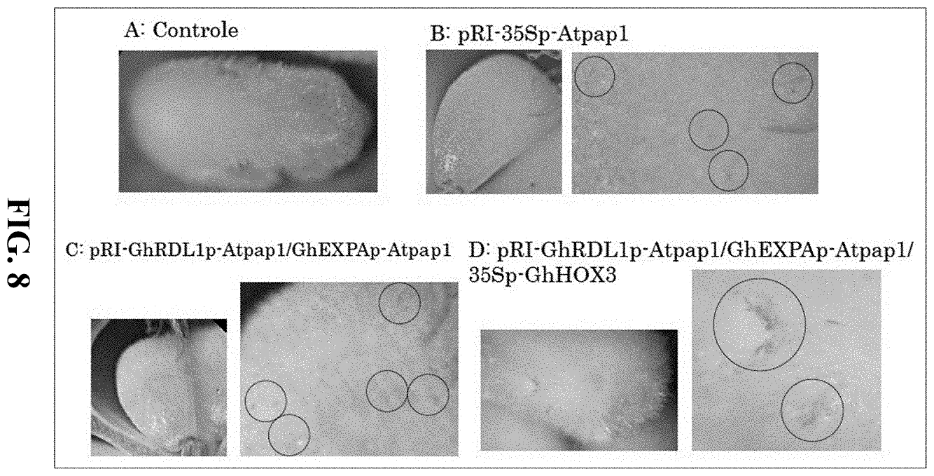

When gold particles not coated with plasmid were introduced into immature seeds, the seeds remained white even after three days or more passed (FIG. 8A). When pRI-35Sp-Atpap1 was introduced, fiber cells in one up to four locations per immature seed were observed to be reddened (FIG. 8B). When pRI-GhRDL1p-Atpap1/GhEXPAp-Atpap1 were introduced, fiber cells in one up to six locations per immature seed were observed to be reddened (FIG. 8C). When pRI-GhRDL1p-Atpap1/GhEXPAp-Atpap1/35Sp-GhHOX3 were introduced, fiber cells in one up to six locations per immature seed were observed to be reddened, and the strongest degree of reddening was observed (FIG. 8D). As a result, increases in the frequency and degree of reddening were observed more in Atpap1 that was controlled by the cottonseed surface-specific promoters GhRDL1p and GhEXPAp and the transcription factor GhHOX3, than in Atpap1 that was controlled by 35Sp, which was a commonly-used constitutive promoter.

Gene Expression in Tobacco Transformants:

Tobacco was infected with agrobacterium containing plasmid pRI-GhRDL1p-Atpap1/GhEXPAp-Atpap1/35Sp-GhHOX3 or pRI-GhRDL1p-Atpap1/GhEXPAp-Atpap1, and transformants were obtained using the regular method.

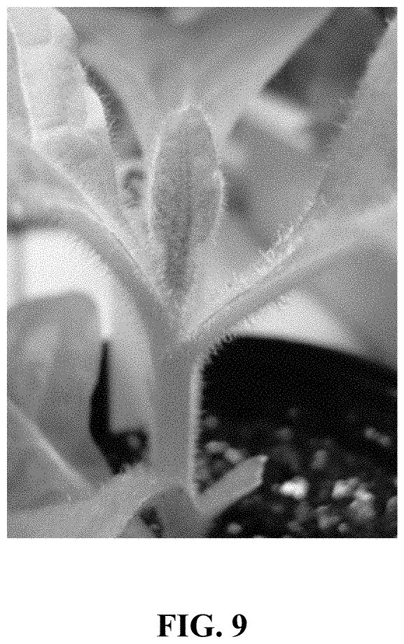

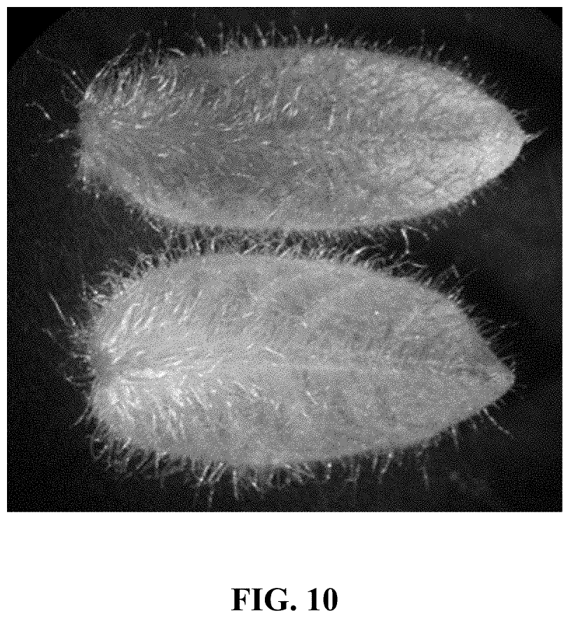

As a result, in the transformant obtained by using pRI-GhRDL1p-Atpap1/GhEXPAp-Atpap1/35Sp-GhHOX3, only the leaf trichome cells were colored red (FIG. 9). The surface cells of the leaves remained green, and especially the trichome cells of young leaves were reddened more distinctly (see the upper one in FIG. 10).

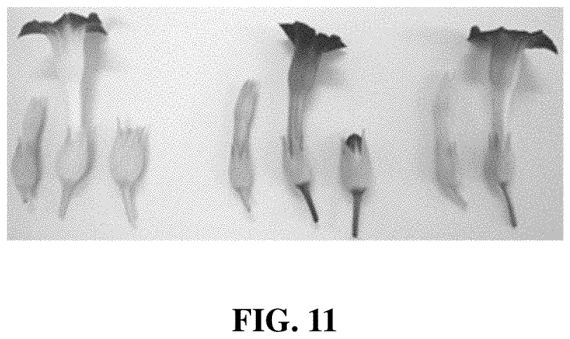

In both of the transformed tobacco obtained by using plasmid pRI-GhRDL1p-Atpap1/GhEXPAp-Atpap1 and the transformed tobacco obtained by using pRI-GhRDL1p-Atpap1/GhEXPAp-Atpap1/35Sp-GhHOX3, the petals were colored (see the center and right ones in FIG. 11. The left one is for comparison).

The present invention is suitable for use in the textile industry, the textile product industry, and the like.

1-14. (canceled)

15. A vector comprising at least one promoter that is expressed specifically in a cottonseed surface and/or a cotton fiber.

16. The vector according to claim 15, further comprising at least one transacting factor gene that is functionally linked to the promoter and activates transcription from the promoter.

17. The vector according to claim 15, wherein the promoter comprises one of:
(1) a GhRDL1 and/or GhEXPA promoter;
(2) a promoter that hybridizes with the GhRDL1 or the GhEXPA promoter under a stringent condition, and that is expressed specifically in the cottonseed surface and/or the cotton fiber; and
(3) a promoter that has 70% or higher homology with the GhRDL1 or the GhEXPA promoter, and that is expressed specifically in the cottonseed surface and/or the cotton fiber.

18. The vector according to claim 16, wherein the transacting factor gene comprises one of:
(1) a DNA that encodes a GhHOX3 protein;
(2) a DNA that encodes a protein including 60% or higher amino acid identity with the GhHOX3 protein and including a transcriptional activation function equivalent to the GhHOX3 protein;
(3) a DNA that encodes a protein including 70% or higher homology with the DNA of (1) and including a transcriptional activation function equivalent to the GhHOX3 protein; and
(4) a DNA that encodes a protein hybridizing with the DNA of (1) under a stringent condition and including a transcriptional activation function equivalent to the GhHOX3 protein.

19. The vector according to claim 15, further comprising at least one transcription factor gene and/or a pigment biosynthetic gene that is functionally linked to the promoter and promotes expression of a pigment biosynthetic gene group.

20. The vector according to claim 19, wherein the transcription factor gene that promotes the expression of the pigment biosynthetic gene group comprises one of:
(1) a DNA that encodes an Atpap1 protein;
(2) a DNA that encodes a protein including 60% or higher amino acid identity with the Atpap1 protein and including a transcriptional activation function equivalent to the Atpap1 protein;
(3) a DNA that encodes a protein including 70% or higher homology with the DNA of (1) and including a transcriptional activation function equivalent to the Atpap1 protein; and
(4) a DNA that encodes a protein hybridizing with the DNA of (1) under a stringent condition and including a transcriptional activation function equivalent to the Atpap1 protein.

21. The vector according to claim 15, wherein the vector further comprises a T-DNA, and the T-DNA includes a border region.

22. A vector comprising any one of SEQ. ID NOs. 1 to 3.

23. A cell, a tissue, a callus, a transgenic plant and/or a seed thereof, transformed with the vector according to claim 15.

24. The cell, the tissue, the callus, the transgenic plant and/or the seed thereof according to claim 23, wherein the transformed cell, tissue, callus, transgenic plant and/or seed thereof is derived from cotton.

25. A clonal plant, a progeny plant and/or a seed thereof, derived from the transgenic plant or the seed thereof according to claim 23.

26. The clonal plant, the progeny plant, and/or the seed thereof according to claim 25, wherein the clonal plant, the progeny plant and/or the seed thereof are derived from cotton.

27. An agrobacterium, wherein the vector according to claim 15 is transformed.

28. A cotton fiber, a fabric, or clothing derived from the transgenic plant according to claim 23.

29. A cotton fiber, a fabric, or clothing derived from the clonal plant or the progeny plant according to claim 25.


SEQUENCE LISTING (17 sequences)

SEQ ID NO: 1 - DNA, 13772 bp, Artificial Sequence
  plasmid pRI-GhRDL1p-Atpap1/35Sp-GhHOX3
gttgccatgttttacggcagtgagagcagagatagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcctggcgaaagggggatgtgctgcaaggcgattaagttgggtaacgccagggttttcccagtcacgacgttgtaaaacgacggccagtgccaagcttgcatgcctgcaggaattagttatgtttggtaaatgaatttgaatacatgcaccaccactctcaatgctcacccacctaagcccatgacataaactgcatttaagtgaaccagtggcagttggagccattatgttggttgaaaaatattgttggcagtgttattcagcagtacttgttctaaggctcctactacatttctcattataaatgtggagaaatcttgttctactttccattccatgcaacactaacttttagattccctttccattgcactgctctttctttctatagaattagtcactcctgttctagtctagactatctttgttccatggagggttcgtccaaagggctgcgaaaaggtgcttggactactgaagaagatagtctcttgagacagtgcattaataagtatggagaaggcaaatggcaccaagttcctgtaagagctgggctaaaccggtgcaggaaaagttgtagattaagatggttgaactatttgaagccaagtatcaagagaggaaaacttagctctgatgaagtcgatcttcttcttcgccttcataggcttctagggaataggtggtctttaattgctggaagattacctggtcggaccgcaaatgacgtcaagaattactggaacactcatctgagtaagaaacatgaaccgtgttgtaagataaagatgaaaaagagagacattacgcccattcctacaacaccggcactaaaaaacaatgtttataagcctcgacctcgatccttcacagttaacaacgactgcaaccatctcaatgccccaccaaaagttgacgttaatcctccatgccttggacttaacatcaataatgtttgtgacaatagtatcatatacaacaaagataagaagaaagaccaactagtgaataatttgattgatggagataatatgtggttagagaaattcctagaggaaagccaagaggtagatattttggttcctgaagcgacgacaacagaaaagggggacaccttggcttttgacgttgatcaactttggagtcttttcgatggagagactgtgaaatttgattagtgtttcgaacatttgtttgcgtttgtggagctcttatgaagatgaagatgaaatatttggtgtgtcaaataaaaagctagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttcgcatataactactgtatgtgctatggtatggactatggaatatgattaaagataaggaattgaattcaagcttgcatgcctgcaggtccccagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctagaatggattgcggaagcggcggcggcggcttaggagcgagtggttccggcggagaccatgattcctccgacctttcacgacggaaaaagccttaccatcgtcacacagctcaccagattcagaggcttgaatcgatgttcaaggaatgtccacacccagacgagaaacaaaggttacagctaagtagggaattgggattagcaccaagacagatcaagttttggtttcaaaataggaggacacaaatgaaggcacaacatgaaagagccgataactctgcacttcgtgctgagaacgataagatccgatgtgaaaacatagccataagagaagcacttaagaatgtgatttgtccttcttgtggtggtcctcctgctaatgaggattcttattttgatgaccagaaaatgagaatggagaatgctcaattaaaggaagagcttgatagagtatccagcattgcagccaagtacatagggagaccaatatcccaactcccaccagtacaacctgttcatatctcttcattagatttcaggatggcgagttttgacggctatggtgtcggcgctggtccttcactcgatctcgatctcctccccggaagttcgtcatcaatgccgaatttaccgttccaaccggtcgttatttcagacattgacaagtccctcatgtctgatatagccgcaaatgcaatggaagaactgcttaggctattacaaaccaatgaaccattgtggattaaatccactaatgatggcaaagatgctcttaatctcgaaagttatgaaagaatcttccctaagcctaataatactcattttaaatctcctaatattagggtcgaagcttctcgagattccggtgttgttattatgaatggcttagctttggttgacatgttcatggattccaacaaatggttggagctatttcccaccattgtgtcaattgccaaaacaatcgaagtgatttcacctgggatgttgggtacacatagtggttctttgcaattgatgtatgaagaactacaggtcttgtctccattagtaccaacacgtgaattttatacacttcgatattgtcaacaaattgaacaagggttatgggctattgtcaatgtttcatatgatttgccacaatttgcttctcaatgtcgatctcatagactcccttctggttgtttgattcaagacatgcctaatggttactccaaggttacttggttagaacatgtggagattgaagacaaaactccgattcatcgattgtatcgagaccttgttcatagtggttcagcatttggagccgaacggtggctcacgacccttcaaaggatgtgcgaacggtttgcttgcttaatggtatcaagcacttcgactcgggatctcggtggagtgattccatctcccgatggcaggagaagcatgatgaagctagctcaaaggatggtaaacaatttctgcacgagcgtcggcacgtcgaatagtcatcggtcgacaacactttccggttcgaatgaaattggtgttcgggtcaccgttcataagagttctgatcccggacaacccaacggtatagttcttagtgcagctactacattttggctccctgttagtccacaaaatgtcttcaatttcttcaaagatgaaagaactagaccacagtgggatgttctatccaacggcaacgcagtccaagaagttgctcatatcgcaaacggatcacatcccggcaactgtatatccgttttacgagcctttaatacaagtcacaacaacatgttaatactacaagagagttgcattgacagttctggttcactggtggtatactgtccagttgatttaccagctattaatgtagcaatgagcggcgaagatccatcttacattccgttacttccgtcaggttttacgataacacccgacggtcatctcgagcaaggtgacggagcatcgacgagttctagtactgggcacggaagatcgtccggtggttcattgattacggtagcgtttcaaatactagtgagcagcttgccatcggcaaaattaaacttggattcggtcacaattgttaataaccttattgctaatactgtacaacaaattaaggctgctttgaattgtcctagttcttgagagctcttatgaagatgaagatgaaatatttggtgtgtcaaataaaaagctagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttcgcatataactactgtatgtgctatggtatggactatggaatatgattaaagataaggaattgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccaggatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccggatctggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactacaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgtatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgggatctctgcggaacaggcggtcgaaggtgccgatatcattacgacagcaacggccgacaagcacaacgccacgatcctgagcgacaatatgatcgggccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaatttgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccccagtacattaaaaacgtccgcaatgtgttattaagttgtctaagcgtcaatttgtttacaccacaatatatcctgccacaagctagctttctgagccgccgattttcctcctcgagttggatgaactcgccgagttcatcgtcaactgaaacagacacggccggattctgtgagacaggttgaaccgcagctctcttccattgataataggtctgaacggaaatacccacgatcttaacggcgtccttcaaggttgcgccgccagcgacctgagcttcgatttgaccgatcttctccagtttttctcggttgctgaggccgcgggttttcggcttcacggatttgaacgatcccgtgcgggctgtttcggctggtgctttctttgctcttctacctctaggagcagccggctcaacttcggcagcagcagtaccgtccggcggattctggatctcttcgtcagccattaatcgtcctctgtgtgggttattgctttgtctgccagctcgatccaagagtcaacgtttgtgcctagggcagtaaataggcagtgctccgcgactacatgcctcggccggcaaaataccgccgcatgtagagcaggctctccttcacgatcaacgatcggcatggggccttcgtgcttgttgagtaatgttatcgctcccatcagagcacgcttggtactccgggaatcggatggtctgtcgatcatccaaaaaacgctcatgttttcaacctattaggtctgtggtcagctgaccacagaccatcctgctccatactcgctaattctagccaaaccgcaacgtcccctgcccgctagccttcaagagcgccattatcatcgggccaagtgaaaacttcccgagttcgctccgccgtgtcagatctcggagatagcccccaggcgaattgatgaagttcgctcgctccaaaatgcacgccatcgctgctgccgcattctccggtcccattgcctcacacgcgtcttggtaagccgacgggctgacccccagcatagaccgaaccaccaccgcagccgacatgaggtcacgccagctagcaaccgcaccgctcggcccataattgccaatggtcgggcacgctttcaggatcatcccgagggggaacgcttttatcggctcgctccttgcccggtctatttcactcggcttagcgccctgctccttttcagagcgaggttcaagttcattaacggattcgggttttgagttctgtatgtgctgctcgctctgggcagcattggtgctattattttctgaattgtctctaatttccaaccggttgattatctcttcctggagcatccacatctcttcgagaattgactctacatcagcaagcgtcggggcgcgtgggattctacccacaagttccacatagacttcctcgacagcttgccagtcgccctccgctccctcttccatagctgccgtaattagcttccgaacgtcccgtcggcaaatcgtcagactttctttggccatcctgaatgctgctcgatcggccatcacctgctgtgccatcatcgctagctcttcggaccgcgcgagaagcggagacaaatcgaagccaaacgcgcgctcgatctgaccagcgccatccttacgagcgtaacgctttccgttggcgctatccttccggacgatcaagcctgactccacgagcatggcgatgtgcctacgcaaagtcgcgccagccatcccatgcgcccgaagggcaagctgagcattcgacgggaagacgatcagctgtgcctcctgacgcaactccgtttccgggtgaaagctcaatagcgcatcaaggacggcaagactgttggactggattccaagtagttccatggccgcggacgcgtccctaaagaccttccacttgtccgctgtcttgccttgtttgatatcggccagcgccgtctggcgccgcacaagcgcaagcgtcattggccgccgcccgaatggcgtcgttacacttcctgtctgcatcatctttcacctttcagcaggcaaaggaaatcagctcaccaaaacggcgctaaaaactcttgacgaggattcgaggaaatgcgattctgttcgcgctagagagacagaagggcttccgcgacggcgacgttgagggggctcttttcttttgcggtttactctccccgtttccgttggttctcagcgtggtacgcttgatacagcgctggcacatgatcgagcacgaaggtcgcaaaatcgggcgtcgccttcctgtcaatcgtgatttccagtttggccttgctctgcgtcacctgtgcaattctggtgccgtctggggtggccatgacctcgggaagtccacgcgcaacccgactgggcttcagactagcgatcaccgccttgaatcgttctgccgatggcagcgcttgaacttcctccgacatagcatatttagccacgtcggccggtgaagaaactttctcaatcagctcggcaagttgttgccaactcggccgtccaacaccaggagcggcaccaatagcatcggtcagttcagaggggagggcgtcaacgagcagaagcatcttggacaaattgctcttgtcgatcgacatcgcggcgatgacaatctctcgagaaaactgcctgttcaggcgatgtgcgaagcgcgccttttcgatgaaggtaagatcttcgcgcacattgttttcctgaccctgtgctacgaccacttgctcgtccgtcagttcgcgaacgaccgccctgaccggaagtccgagttctgaaacggcgcgtagccggcggtggccgaaggcaacctgatatcggcccggctggctcggatgcggtcgcacaaggattgggacttgctgtccttgttcccggatcgaagtaaggagcccgtcaatgtcccctcgcatacgatcctgcacgaaagacggttctattgacgaggcatccaactctatcactgcctgaccttcagcgagacgccgctcgatctcttcggcacggctaagacgatcgttttgctctcgcagtgcgttaccaatgttcgctgtgagcttcgttgccggatcgcgctccttccttgttacgccgaggagcggcatggagcggttctttgccgtcctattgtcggcgggcgacgtctcaggggcgtcagttgagacgccaaggatgtgcttccggctcatgtgggcctaccccatgcttttttgatcagtgtttcgatctcgtcgttgacggcgttcatcgcctccaaggctcgatcataggtcgagcgcgtgaacaggccacgctccacttcgaatagagtctggtttgtcaggccagcgtccgaaaccgcggtggttttaagcatcggaaaattgaggacattttcgccaaaaatcgaccgcagataacctaccatttggttctgtggtccgtcgctcggttcgaaacgggttatcagatagcgcatccaattaaacttgaacttggcgccagcattctcgatttcacgcaaaaggttcgatgtcattgccagaaactggttcatcgacatcacatccagcatctgcggatggaccgtgacaagaatggacgtcgccgcagtcaatgcggatagcgtgagatacccaagctggggagggcagtcgatgaccacgacgtcatagttatccgcgatatcttcaattacttggctgatgcgaccataaaagagcgtgtcgccctctttgcggttcatcagcgcgcgtggcgtatcgtgttcaaactccatcagctcaaggttaccaggaatcaggtggaggtcgggaatgtaagtccctcggacgactcgttcgattgccacctgctcatcatcataccttatagcgccgtagagcgtttcgttcgggccaacgtccgtctccggttggctcccaaagagtgcagaaaggctcgcttgaggatcgagatcaatggccaagactcgatatccgcgcatagcgaggtactgcgccagatgcgcggcggtggtggtcttacccgacccacctttgaaattcatcacagagataacctgaagctgctcgccgcctcgacgatgtggcaggtagcgccggttcccgcggccgacctgatccatatacttccgaatcacatggatatcttcaattgagaacattcgcctgccacctgggctcatgctaacattcaactctggcatctcagacgcggtctgccgtaaatatgactcgccaacgccgagcagcttggacgcctccgatggcccgaatgttcgaatacccttctcggaatgcggcgggaaaaccttaagatgatgtgcttgaagttggctcgagagggcatcggcatgacgctccatcaaggccgtcaaccctacaactacaggcgctgcttttaggacagacttcgccatctcaaacccattccttgccagtggcgatatttttcgcgaaactggaaaagttccgccgctggcaattagcgccgattctgctgtttgggcaagagcttttaggttaacagaaggttaacgccctcaggtcgaaaaactccacccaactgttatttgtatttatttccaatgccttagagagattgccatttgaatatgttcatgtattgttttagtgataatcctacaatcgtaacccaaaaagaggtcgccctctgcgcgccgtcgtccaatataggcgaagtcacccttgcgactcaggcggattctaccttgtaggatcgagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctca

SEQ ID NO: 2 - DNA, 15470 bp, Artificial Sequence
  pRI-GhEXPAp-Atpap1/35Sp-GhHOX3
gttgccatgttttacggcagtgagagcagagatagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcctggcgaaagggggatgtgctgcaaggcgattaagttgggtaacgccagggttttcccagtcacgacgttgtaaaacgacggccagtgccaagcttgcatgcctgcaggtttaagcaaaaaattaatagtaagttctatattagaaaattatttaatttcaatcaaacaatgcttaaataaattaaaaagttatataaaatataaaatttagacactataatattaaaataattcagacaactattatcatttttaataagttttctaaaataatgtatgcgaaaaacacttactttattaatacttaaataaatataatttattatatttacaaataaatatttgtatttattcatatgcaaatttttttaaaaatataaatattaaaaaaattatataatttcataaaagtggaaattaattattaaaaaatgaaaattaaataagattaattcattttcctaagaaatataaaaattctaggagctaacatatttaataatattatcatatttctaaaattagttaatattttaaattatggagactaaataagaaaattaaaaaagtgaaagcgctttataaaataaagtaaaaatattgaggggtttatgttacaaataacccaatcgcgtttcccatagagaaaaggaattttctttcttttattttcatgtcattttttcatacaatttctttggtcacttaaattctcaataaaattagaaaacagccctcaaaaataatattgatacgttgaaagatttatcaactttcgaccatcgacactttaaaaaaattagaaagttataatttttttttcaaaaaaactataatacctctctagttttagctaattaaattattattattttattattttattgttattaaaagtgtaacttgcactcaactattagtaagtttacgttttgatcacttaatttcagaaagttaaaaaatggtctttgaactattcgaaaattttcatttaagttactggaatatttaaaagtttttatttaagtcaccgggctattaagtttttttttaaaaattcgattagcaagttccaagctacgattcgataagtgatacaatggatttatacttattgacaaatagaatatacattaggtccaagttgatattacggtcagtgttgaaaatcgaaaaaaaatatttggattttgattcataaatttatgacttcaaagctggttcatgaaaaagaactaaagtgtaagagggaaggaaaaaaatatcttttgattggcacaaacagtgcgaacaaagaagaccacacaataacaattttaacaatatactaatttaaatgaaaaattttcaataatttaataagttaaccgaggaaaacttactaagagttagttacccctgttaaaataactttcatgaagtaatagaaacttttagtacgtatcatcttatatagaacaatttctattttcagaaagtcaagaaaattgtattctagaaaatggcgacttcttcaccttcagtccttccctgatcggcgcttgtgaaaaacgaaaaacctgagtctgattggctgactgaaaatgaacctactcatcaccattcactattaccaacttcaaatgataggggaattaactggtaaagtgtaactccaccgatggttgaggtggttggctggagttaaatgagatttttttagttttgtttcaagtggcttcaattgcaagcaattaggagactgcgctggaataacccctcgctcaaccttccgccattgttatggtttaattaaacattatgtttccatccatctatatttatatccattaaaacaagtcgttgagcaaataatggatactggataccatcatatctatgattaaaattttgcatgtgcccttttaatgtatagcttaagccttaattatcctccaaatttgtactctttcaccacttaattagctacgtacggtacttagcgttgcttgtcatcttctgtactacaaactctttctcattttgtataaatagctatacactttttctctcctcaaatcaataaggttaggtcagccaattgtttgagctagctagctcttactcaatctagactatctttgttccatggagggttcgtccaaagggctgcgaaaaggtgcttggactactgaagaagatagtctcttgagacagtgcattaataagtatggagaaggcaaatggcaccaagttcctgtaagagctgggctaaaccggtgcaggaaaagttgtagattaagatggttgaactatttgaagccaagtatcaagagaggaaaacttagctctgatgaagtcgatcttcttcttcgccttcataggcttctagggaataggtggtctttaattgctggaagattacctggtcggaccgcaaatgacgtcaagaattactggaacactcatctgagtaagaaacatgaaccgtgttgtaagataaagatgaaaaagagagacattacgcccattcctacaacaccggcactaaaaaacaatgtttataagcctcgacctcgatccttcacagttaacaacgactgcaaccatctcaatgccccaccaaaagttgacgttaatcctccatgccttggacttaacatcaataatgtttgtgacaatagtatcatatacaacaaagataagaagaaagaccaactagtgaataatttgattgatggagataatatgtggttagagaaattcctagaggaaagccaagaggtagatattttggttcctgaagcgacgacaacagaaaagggggacaccttggcttttgacgttgatcaactttggagtcttttcgatggagagactgtgaaatttgattagtgtttcgaacatttgtttgcgtttgtggagctcttatgaagatgaagatgaaatatttggtgtgtcaaataaaaagctagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttcgcatataactactgtatgtgctatggtatggactatggaatatgattaaagataaggaattgaattcaagcttgcatgcctgcaggtccccagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctagaatggattgcggaagcggcggcggcggcttaggagcgagtggttccggcggagaccatgattcctccgacctttcacgacggaaaaagccttaccatcgtcacacagctcaccagattcagaggcttgaatcgatgttcaaggaatgtccacacccagacgagaaacaaaggttacagctaagtagggaattgggattagcaccaagacagatcaagttttggtttcaaaataggaggacacaaatgaaggcacaacatgaaagagccgataactctgcacttcgtgctgagaacgataagatccgatgtgaaaacatagccataagagaagcacttaagaatgtgatttgtccttcttgtggtggtcctcctgctaatgaggattcttattttgatgaccagaaaatgagaatggagaatgctcaattaaaggaagagcttgatagagtatccagcattgcagccaagtacatagggagaccaatatcccaactcccaccagtacaacctgttcatatctcttcattagatttcaggatggcgagttttgacggctatggtgtcggcgctggtccttcactcgatctcgatctcctccccggaagttcgtcatcaatgccgaatttaccgttccaaccggtcgttatttcagacattgacaagtccctcatgtctgatatagccgcaaatgcaatggaagaactgcttaggctattacaaaccaatgaaccattgtggattaaatccactaatgatggcaaagatgctcttaatctcgaaagttatgaaagaatcttccctaagcctaataatactcattttaaatctcctaatattagggtcgaagcttctcgagattccggtgttgttattatgaatggcttagctttggttgacatgttcatggattccaacaaatggttggagctatttcccaccattgtgtcaattgccaaaacaatcgaagtgatttcacctgggatgttgggtacacatagtggttctttgcaattgatgtatgaagaactacaggtcttgtctccattagtaccaacacgtgaattttatacacttcgatattgtcaacaaattgaacaagggttatgggctattgtcaatgtttcatatgatttgccacaatttgcttctcaatgtcgatctcatagactcccttctggttgtttgattcaagacatgcctaatggttactccaaggttacttggttagaacatgtggagattgaagacaaaactccgattcatcgattgtatcgagaccttgttcatagtggttcagcatttggagccgaacggtggctcacgacccttcaaaggatgtgcgaacggtttgcttgcttaatggtatcaagcacttcgactcgggatctcggtggagtgattccatctcccgatggcaggagaagcatgatgaagctagctcaaaggatggtaaacaatttctgcacgagcgtcggcacgtcgaatagtcatcggtcgacaacactttccggttcgaatgaaattggtgttcgggtcaccgttcataagagttctgatcccggacaacccaacggtatagttcttagtgcagctactacattttggctccctgttagtccacaaaatgtcttcaatttcttcaaagatgaaagaactagaccacagtgggatgttctatccaacggcaacgcagtccaagaagttgctcatatcgcaaacggatcacatcccggcaactgtatatccgttttacgagcctttaatacaagtcacaacaacatgttaatactacaagagagttgcattgacagttctggttcactggtggtatactgtccagttgatttaccagctattaatgtagcaatgagcggcgaagatccatcttacattccgttacttccgtcaggttttacgataacacccgacggtcatctcgagcaaggtgacggagcatcgacgagttctagtactgggcacggaagatcgtccggtggttcattgattacggtagcgtttcaaatactagtgagcagcttgccatcggcaaaattaaacttggattcggtcacaattgttaataaccttattgctaatactgtacaacaaattaaggctgctttgaattgtcctagttcttgagagctcttatgaagatgaagatgaaatatttggtgtgtcaaataaaaagctagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttcgcatataactactgtatgtgctatggtatggactatggaatatgattaaagataaggaattgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccaggatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccggatctggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactacaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgtatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgggatctctgcggaacaggcggtcgaaggtgccgatatcattacgacagcaacggccgacaagcacaacgccacgatcctgagcgacaatatgatcgggccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaatttgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccccagtacattaaaaacgtccgcaatgtgttattaagttgtctaagcgtcaatttgtttacaccacaatatatcctgccacaagctagctttctgagccgccgattttcctcctcgagttggatgaactcgccgagttcatcgtcaactgaaacagacacggccggattctgtgagacaggttgaaccgcagctctcttccattgataataggtctgaacggaaatacccacgatcttaacggcgtccttcaaggttgcgccgccagcgacctgagcttcgatttgaccgatcttctccagtttttctcggttgctgaggccgcgggttttcggcttcacggatttgaacgatcccgtgcgggctgtttcggctggtgctttctttgctcttctacctctaggagcagccggctcaacttcggcagcagcagtaccgtccggcggattctggatctcttcgtcagccattaatcgtcctctgtgtgggttattgctttgtctgccagctcgatccaagagtcaacgtttgtgcctagggcagtaaataggcagtgctccgcgactacatgcctcggccggcaaaataccgccgcatgtagagcaggctctccttcacgatcaacgatcggcatggggccttcgtgcttgttgagtaatgttatcgctcccatcagagcacgcttggtactccgggaatcggatggtctgtcgatcatccaaaaaacgctcatgttttcaacctattaggtctgtggtcagctgaccacagaccatcctgctccatactcgctaattctagccaaaccgcaacgtcccctgcccgctagccttcaagagcgccattatcatcgggccaagtgaaaacttcccgagttcgctccgccgtgtcagatctcggagatagcccccaggcgaattgatgaagttcgctcgctccaaaatgcacgccatcgctgctgccgcattctccggtcccattgcctcacacgcgtcttggtaagccgacgggctgacccccagcatagaccgaaccaccaccgcagccgacatgaggtcacgccagctagcaaccgcaccgctcggcccataattgccaatggtcgggcacgctttcaggatcatcccgagggggaacgcttttatcggctcgctccttgcccggtctatttcactcggcttagcgccctgctccttttcagagcgaggttcaagttcattaacggattcgggttttgagttctgtatgtgctgctcgctctgggcagcattggtgctattattttctgaattgtctctaatttccaaccggttgattatctcttcctggagcatccacatctcttcgagaattgactctacatcagcaagcgtcggggcgcgtgggattctacccacaagttccacatagacttcctcgacagcttgccagtcgccctccgctccctcttccatagctgccgtaattagcttccgaacgtcccgtcggcaaatcgtcagactttctttggccatcctgaatgctgctcgatcggccatcacctgctgtgccatcatcgctagctcttcggaccgcgcgagaagcggagacaaatcgaagccaaacgcgcgctcgatctgaccagcgccatccttacgagcgtaacgctttccgttggcgctatccttccggacgatcaagcctgactccacgagcatggcgatgtgcctacgcaaagtcgcgccagccatcccatgcgcccgaagggcaagctgagcattcgacgggaagacgatcagctgtgcctcctgacgcaactccgtttccgggtgaaagctcaatagcgcatcaaggacggcaagactgttggactggattccaagtagttccatggccgcggacgcgtccctaaagaccttccacttgtccgctgtcttgccttgtttgatatcggccagcgccgtctggcgccgcacaagcgcaagcgtcattggccgccgcccgaatggcgtcgttacacttcctgtctgcatcatctttcacctttcagcaggcaaaggaaatcagctcaccaaaacggcgctaaaaactcttgacgaggattcgaggaaatgcgattctgttcgcgctagagagacagaagggcttccgcgacggcgacgttgagggggctcttttcttttgcggtttactctccccgtttccgttggttctcagcgtggtacgcttgatacagcgctggcacatgatcgagcacgaaggtcgcaaaatcgggcgtcgccttcctgtcaatcgtgatttccagtttggccttgctctgcgtcacctgtgcaattctggtgccgtctggggtggccatgacctcgggaagtccacgcgcaacccgactgggcttcagactagcgatcaccgccttgaatcgttctgccgatggcagcgcttgaacttcctccgacatagcatatttagccacgtcggccggtgaagaaactttctcaatcagctcggcaagttgttgccaactcggccgtccaacaccaggagcggcaccaatagcatcggtcagttcagaggggagggcgtcaacgagcagaagcatcttggacaaattgctcttgtcgatcgacatcgcggcgatgacaatctctcgagaaaactgcctgttcaggcgatgtgcgaagcgcgccttttcgatgaaggtaagatcttcgcgcacattgttttcctgaccctgtgctacgaccacttgctcgtccgtcagttcgcgaacgaccgccctgaccggaagtccgagttctgaaacggcgcgtagccggcggtggccgaaggcaacctgatatcggcccggctggctcggatgcggtcgcacaaggattgggacttgctgtccttgttcccggatcgaagtaaggagcccgtcaatgtcccctcgcatacgatcctgcacgaaagacggttctattgacgaggcatccaactctatcactgcctgaccttcagcgagacgccgctcgatctcttcggcacggctaagacgatcgttttgctctcgcagtgcgttaccaatgttcgctgtgagcttcgttgccggatcgcgctccttccttgttacgccgaggagcggcatggagcggttctttgccgtcctattgtcggcgggcgacgtctcaggggcgtcagttgagacgccaaggatgtgcttccggctcatgtgggcctaccccatgcttttttgatcagtgtttcgatctcgtcgttgacggcgttcatcgcctccaaggctcgatcataggtcgagcgcgtgaacaggccacgctccacttcgaatagagtctggtttgtcaggccagcgtccgaaaccgcggtggttttaagcatcggaaaattgaggacattttcgccaaaaatcgaccgcagataacctaccatttggttctgtggtccgtcgctcggttcgaaacgggttatcagatagcgcatccaattaaacttgaacttggcgccagcattctcgatttcacgcaaaaggttcgatgtcattgccagaaactggttcatcgacatcacatccagcatctgcggatggaccgtgacaagaatggacgtcgccgcagtcaatgcggatagcgtgagatacccaagctggggagggcagtcgatgaccacgacgtcatagttatccgcgatatcttcaattacttggctgatgcgaccataaaagagcgtgtcgccctctttgcggttcatcagcgcgcgtggcgtatcgtgttcaaactccatcagctcaaggttaccaggaatcaggtggaggtcgggaatgtaagtccctcggacgactcgttcgattgccacctgctcatcatcataccttatagcgccgtagagcgtttcgttcgggccaacgtccgtctccggttggctcccaaagagtgcagaaaggctcgcttgaggatcgagatcaatggccaagactcgatatccgcgcatagcgaggtactgcgccagatgcgcggcggtggtggtcttacccgacccacctttgaaattcatcacagagataacctgaagctgctcgccgcctcgacgatgtggcaggtagcgccggttcccgcggccgacctgatccatatacttccgaatcacatggatatcttcaattgagaacattcgcctgccacctgggctcatgctaacattcaactctggcatctcagacgcggtctgccgtaaatatgactcgccaacgccgagcagcttggacgcctccgatggcccgaatgttcgaatacccttctcggaatgcggcgggaaaaccttaagatgatgtgcttgaagttggctcgagagggcatcggcatgacgctccatcaaggccgtcaaccctacaactacaggcgctgcttttaggacagacttcgccatctcaaacccattccttgccagtggcgatatttttcgcgaaactggaaaagttccgccgctggcaattagcgccgattctgctgtttgggcaagagcttttaggttaacagaaggttaacgccctcaggtcgaaaaactccacccaactgttatttgtatttatttccaatgccttagagagattgccatttgaatatgttcatgtattgttttagtgataatcctacaatcgtaacccaaaaagaggtcgccctctgcgcgccgtcgtccaatataggcgaagtcacccttgcgactcaggcggattctaccttgtaggatcgagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctca

SEQ ID NO: 3 - DNA, 16845 bp, Artificial Sequence
  plasmid pRI-GhRDL1p-Atpap1/GhEXPAp-Atpap1/35Sp- GhHOX3
gttgccatgttttacggcagtgagagcagagatagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcctggcgaaagggggatgtgctgcaaggcgattaagttgggtaacgccagggttttcccagtcacgacgttgtaaaacgacggccagtgccaagcttgcatgcctgcaggaattagttatgtttggtaaatgaatttgaatacatgcaccaccactctcaatgctcacccacctaagcccatgacataaactgcatttaagtgaaccagtggcagttggagccattatgttggttgaaaaatattgttggcagtgttattcagcagtacttgttctaaggctcctactacatttctcattataaatgtggagaaatcttgttctactttccattccatgcaacactaacttttagattccctttccattgcactgctctttctttctatagaattagtcactcctgttctagtctagactatctttgttccatggagggttcgtccaaagggctgcgaaaaggtgcttggactactgaagaagatagtctcttgagacagtgcattaataagtatggagaaggcaaatggcaccaagttcctgtaagagctgggctaaaccggtgcaggaaaagttgtagattaagatggttgaactatttgaagccaagtatcaagagaggaaaacttagctctgatgaagtcgatcttcttcttcgccttcataggcttctagggaataggtggtctttaattgctggaagattacctggtcggaccgcaaatgacgtcaagaattactggaacactcatctgagtaagaaacatgaaccgtgttgtaagataaagatgaaaaagagagacattacgcccattcctacaacaccggcactaaaaaacaatgtttataagcctcgacctcgatccttcacagttaacaacgactgcaaccatctcaatgccccaccaaaagttgacgttaatcctccatgccttggacttaacatcaataatgtttgtgacaatagtatcatatacaacaaagataagaagaaagaccaactagtgaataatttgattgatggagataatatgtggttagagaaattcctagaggaaagccaagaggtagatattttggttcctgaagcgacgacaacagaaaagggggacaccttggcttttgacgttgatcaactttggagtcttttcgatggagagactgtgaaatttgattagtgtttcgaacatttgtttgcgtttgtggagctcttatgaagatgaagatgaaatatttggtgtgtcaaataaaaagctagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttcgcatataactactgtatgtgctatggtatggactatggaatatgattaaagataaggaattaagcttgcatgcctgcaggtttaagcaaaaaattaatagtaagttctatattagaaaattatttaatttcaatcaaacaatgcttaaataaattaaaaagttatataaaatataaaatttagacactataatattaaaataattcagacaactattatcatttttaataagttttctaaaataatgtatgcgaaaaacacttactttattaatacttaaataaatataatttattatatttacaaataaatatttgtatttattcatatgcaaatttttttaaaaatataaatattaaaaaaattatataatttcataaaagtggaaattaattattaaaaaatgaaaattaaataagattaattcattttcctaagaaatataaaaattctaggagctaacatatttaataatattatcatatttctaaaattagttaatattttaaattatggagactaaataagaaaattaaaaaagtgaaagcgctttataaaataaagtaaaaatattgaggggtttatgttacaaataacccaatcgcgtttcccatagagaaaaggaattttctttcttttattttcatgtcattttttcatacaatttctttggtcacttaaattctcaataaaattagaaaacagccctcaaaaataatattgatacgttgaaagatttatcaactttcgaccatcgacactttaaaaaaattagaaagttataatttttttttcaaaaaaactataatacctctctagttttagctaattaaattattattattttattattttattgttattaaaagtgtaacttgcactcaactattagtaagtttacgttttgatcacttaatttcagaaagttaaaaaatggtctttgaactattcgaaaattttcatttaagttactggaatatttaaaagtttttatttaagtcaccgggctattaagtttttttttaaaaattcgattagcaagttccaagctacgattcgataagtgatacaatggatttatacttattgacaaatagaatatacattaggtccaagttgatattacggtcagtgttgaaaatcgaaaaaaaatatttggattttgattcataaatttatgacttcaaagctggttcatgaaaaagaactaaagtgtaagagggaaggaaaaaaatatcttttgattggcacaaacagtgcgaacaaagaagaccacacaataacaattttaacaatatactaatttaaatgaaaaattttcaataatttaataagttaaccgaggaaaacttactaagagttagttacccctgttaaaataactttcatgaagtaatagaaacttttagtacgtatcatcttatatagaacaatttctattttcagaaagtcaagaaaattgtattctagaaaatggcgacttcttcaccttcagtccttccctgatcggcgcttgtgaaaaacgaaaaacctgagtctgattggctgactgaaaatgaacctactcatcaccattcactattaccaacttcaaatgataggggaattaactggtaaagtgtaactccaccgatggttgaggtggttggctggagttaaatgagatttttttagttttgtttcaagtggcttcaattgcaagcaattaggagactgcgctggaataacccctcgctcaaccttccgccattgttatggtttaattaaacattatgtttccatccatctatatttatatccattaaaacaagtcgttgagcaaataatggatactggataccatcatatctatgattaaaattttgcatgtgcccttttaatgtatagcttaagccttaattatcctccaaatttgtactctttcaccacttaattagctacgtacggtacttagcgttgcttgtcatcttctgtactacaaactctttctcattttgtataaatagctatacactttttctctcctcaaatcaataaggttaggtcagccaattgtttgagctagctagctcttactcaatctagactatctttgttccatggagggttcgtccaaagggctgcgaaaaggtgcttggactactgaagaagatagtctcttgagacagtgcattaataagtatggagaaggcaaatggcaccaagttcctgtaagagctgggctaaaccggtgcaggaaaagttgtagattaagatggttgaactatttgaagccaagtatcaagagaggaaaacttagctctgatgaagtcgatcttcttcttcgccttcataggcttctagggaataggtggtctttaattgctggaagattacctggtcggaccgcaaatgacgtcaagaattactggaacactcatctgagtaagaaacatgaaccgtgttgtaagataaagatgaaaaagagagacattacgcccattcctacaacaccggcactaaaaaacaatgtttataagcctcgacctcgatccttcacagttaacaacgactgcaaccatctcaatgccccaccaaaagttgacgttaatcctccatgccttggacttaacatcaataatgtttgtgacaatagtatcatatacaacaaagataagaagaaagaccaactagtgaataatttgattgatggagataatatgtggttagagaaattcctagaggaaagccaagaggtagatattttggttcctgaagcgacgacaacagaaaagggggacaccttggcttttgacgttgatcaactttggagtcttttcgatggagagactgtgaaatttgattagtgtttcgaacatttgtttgcgtttgtggagctcttatgaagatgaagatgaaatatttggtgtgtcaaataaaaagctagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttcgcatataactactgtatgtgctatggtatggactatggaatatgattaaagataaggaattgaattcaagcttgcatgcctgcaggtccccagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctagaatggattgcggaagcggcggcggcggcttaggagcgagtggttccggcggagaccatgattcctccgacctttcacgacggaaaaagccttaccatcgtcacacagctcaccagattcagaggcttgaatcgatgttcaaggaatgtccacacccagacgagaaacaaaggttacagctaagtagggaattgggattagcaccaagacagatcaagttttggtttcaaaataggaggacacaaatgaaggcacaacatgaaagagccgataactctgcacttcgtgctgagaacgataagatccgatgtgaaaacatagccataagagaagcacttaagaatgtgatttgtccttcttgtggtggtcctcctgctaatgaggattcttattttgatgaccagaaaatgagaatggagaatgctcaattaaaggaagagcttgatagagtatccagcattgcagccaagtacatagggagaccaatatcccaactcccaccagtacaacctgttcatatctcttcattagatttcaggatggcgagttttgacggctatggtgtcggcgctggtccttcactcgatctcgatctcctccccggaagttcgtcatcaatgccgaatttaccgttccaaccggtcgttatttcagacattgacaagtccctcatgtctgatatagccgcaaatgcaatggaagaactgcttaggctattacaaaccaatgaaccattgtggattaaatccactaatgatggcaaagatgctcttaatctcgaaagttatgaaagaatcttccctaagcctaataatactcattttaaatctcctaatattagggtcgaagcttctcgagattccggtgttgttattatgaatggcttagctttggttgacatgttcatggattccaacaaatggttggagctatttcccaccattgtgtcaattgccaaaacaatcgaagtgatttcacctgggatgttgggtacacatagtggttctttgcaattgatgtatgaagaactacaggtcttgtctccattagtaccaacacgtgaattttatacacttcgatattgtcaacaaattgaacaagggttatgggctattgtcaatgtttcatatgatttgccacaatttgcttctcaatgtcgatctcatagactcccttctggttgtttgattcaagacatgcctaatggttactccaaggttacttggttagaacatgtggagattgaagacaaaactccgattcatcgattgtatcgagaccttgttcatagtggttcagcatttggagccgaacggtggctcacgacccttcaaaggatgtgcgaacggtttgcttgcttaatggtatcaagcacttcgactcgggatctcggtggagtgattccatctcccgatggcaggagaagcatgatgaagctagctcaaaggatggtaaacaatttctgcacgagcgtcggcacgtcgaatagtcatcggtcgacaacactttccggttcgaatgaaattggtgttcgggtcaccgttcataagagttctgatcccggacaacccaacggtatagttcttagtgcagctactacattttggctccctgttagtccacaaaatgtcttcaatttcttcaaagatgaaagaactagaccacagtgggatgttctatccaacggcaacgcagtccaagaagttgctcatatcgcaaacggatcacatcccggcaactgtatatccgttttacgagcctttaatacaagtcacaacaacatgttaatactacaagagagttgcattgacagttctggttcactggtggtatactgtccagttgatttaccagctattaatgtagcaatgagcggcgaagatccatcttacattccgttacttccgtcaggttttacgataacacccgacggtcatctcgagcaaggtgacggagcatcgacgagttctagtactgggcacggaagatcgtccggtggttcattgattacggtagcgtttcaaatactagtgagcagcttgccatcggcaaaattaaacttggattcggtcacaattgttaataaccttattgctaatactgtacaacaaattaaggctgctttgaattgtcctagttcttgagagctcttatgaagatgaagatgaaatatttggtgtgtcaaataaaaagctagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttcgcatataactactgtatgtgctatggtatggactatggaatatgattaaagataaggaattgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccaggatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccggatctggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactacaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgtatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgggatctctgcggaacaggcggtcgaaggtgccgatatcattacgacagcaacggccgacaagcacaacgccacgatcctgagcgacaatatgatcgggccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaatttgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccccagtacattaaaaacgtccgcaatgtgttattaagttgtctaagcgtcaatttgtttacaccacaatatatcctgccacaagctagctttctgagccgccgattttcctcctcgagttggatgaactcgccgagttcatcgtcaactgaaacagacacggccggattctgtgagacaggttgaaccgcagctctcttccattgataataggtctgaacggaaatacccacgatcttaacggcgtccttcaaggttgcgccgccagcgacctgagcttcgatttgaccgatcttctccagtttttctcggttgctgaggccgcgggttttcggcttcacggatttgaacgatcccgtgcgggctgtttcggctggtgctttctttgctcttctacctctaggagcagccggctcaacttcggcagcagcagtaccgtccggcggattctggatctcttcgtcagccattaatcgtcctctgtgtgggttattgctttgtctgccagctcgatccaagagtcaacgtttgtgcctagggcagtaaataggcagtgctccgcgactacatgcctcggccggcaaaataccgccgcatgtagagcaggctctccttcacgatcaacgatcggcatggggccttcgtgcttgttgagtaatgttatcgctcccatcagagcacgcttggtactccgggaatcggatggtctgtcgatcatccaaaaaacgctcatgttttcaacctattaggtctgtggtcagctgaccacagaccatcctgctccatactcgctaattctagccaaaccgcaacgtcccctgcccgctagccttcaagagcgccattatcatcgggccaagtgaaaacttcccgagttcgctccgccgtgtcagatctcggagatagcccccaggcgaattgatgaagttcgctcgctccaaaatgcacgccatcgctgctgccgcattctccggtcccattgcctcacacgcgtcttggtaagccgacgggctgacccccagcatagaccgaaccaccaccgcagccgacatgaggtcacgccagctagcaaccgcaccgctcggcccataattgccaatggtcgggcacgctttcaggatcatcccgagggggaacgcttttatcggctcgctccttgcccggtctatttcactcggcttagcgccctgctccttttcagagcgaggttcaagttcattaacggattcgggttttgagttctgtatgtgctgctcgctctgggcagcattggtgctattattttctgaattgtctctaatttccaaccggttgattatctcttcctggagcatccacatctcttcgagaattgactctacatcagcaagcgtcggggcgcgtgggattctacccacaagttccacatagacttcctcgacagcttgccagtcgccctccgctccctcttccatagctgccgtaattagcttccgaacgtcccgtcggcaaatcgtcagactttctttggccatcctgaatgctgctcgatcggccatcacctgctgtgccatcatcgctagctcttcggaccgcgcgagaagcggagacaaatcgaagccaaacgcgcgctcgatctgaccagcgccatccttacgagcgtaacgctttccgttggcgctatccttccggacgatcaagcctgactccacgagcatggcgatgtgcctacgcaaagtcgcgccagccatcccatgcgcccgaagggcaagctgagcattcgacgggaagacgatcagctgtgcctcctgacgcaactccgtttccgggtgaaagctcaatagcgcatcaaggacggcaagactgttggactggattccaagtagttccatggccgcggacgcgtccctaaagaccttccacttgtccgctgtcttgccttgtttgatatcggccagcgccgtctggcgccgcacaagcgcaagcgtcattggccgccgcccgaatggcgtcgttacacttcctgtctgcatcatctttcacctttcagcaggcaaaggaaatcagctcaccaaaacggcgctaaaaactcttgacgaggattcgaggaaatgcgattctgttcgcgctagagagacagaagggcttccgcgacggcgacgttgagggggctcttttcttttgcggtttactctccccgtttccgttggttctcagcgtggtacgcttgatacagcgctggcacatgatcgagcacgaaggtcgcaaaatcgggcgtcgccttcctgtcaatcgtgatttccagtttggccttgctctgcgtcacctgtgcaattctggtgccgtctggggtggccatgacctcgggaagtccacgcgcaacccgactgggcttcagactagcgatcaccgccttgaatcgttctgccgatggcagcgcttgaacttcctccgacatagcatatttagccacgtcggccggtgaagaaactttctcaatcagctcggcaagttgttgccaactcggccgtccaacaccaggagcggcaccaatagcatcggtcagttcagaggggagggcgtcaacgagcagaagcatcttggacaaattgctcttgtcgatcgacatcgcggcgatgacaatctctcgagaaaactgcctgttcaggcgatgtgcgaagcgcgccttttcgatgaaggtaagatcttcgcgcacattgttttcctgaccctgtgctacgaccacttgctcgtccgtcagttcgcgaacgaccgccctgaccggaagtccgagttctgaaacggcgcgtagccggcggtggccgaaggcaacctgatatcggcccggctggctcggatgcggtcgcacaaggattgggacttgctgtccttgttcccggatcgaagtaaggagcccgtcaatgtcccctcgcatacgatcctgcacgaaagacggttctattgacgaggcatccaactctatcactgcctgaccttcagcgagacgccgctcgatctcttcggcacggctaagacgatcgttttgctctcgcagtgcgttaccaatgttcgctgtgagcttcgttgccggatcgcgctccttccttgttacgccgaggagcggcatggagcggttctttgccgtcctattgtcggcgggcgacgtctcaggggcgtcagttgagacgccaaggatgtgcttccggctcatgtgggcctaccccatgcttttttgatcagtgtttcgatctcgtcgttgacggcgttcatcgcctccaaggctcgatcataggtcgagcgcgtgaacaggccacgctccacttcgaatagagtctggtttgtcaggccagcgtccgaaaccgcggtggttttaagcatcggaaaattgaggacattttcgccaaaaatcgaccgcagataacctaccatttggttctgtggtccgtcgctcggttcgaaacgggttatcagatagcgcatccaattaaacttgaacttggcgccagcattctcgatttcacgcaaaaggttcgatgtcattgccagaaactggttcatcgacatcacatccagcatctgcggatggaccgtgacaagaatggacgtcgccgcagtcaatgcggatagcgtgagatacccaagctggggagggcagtcgatgaccacgacgtcatagttatccgcgatatcttcaattacttggctgatgcgaccataaaagagcgtgtcgccctctttgcggttcatcagcgcgcgtggcgtatcgtgttcaaactccatcagctcaaggttaccaggaatcaggtggaggtcgggaatgtaagtccctcggacgactcgttcgattgccacctgctcatcatcataccttatagcgccgtagagcgtttcgttcgggccaacgtccgtctccggttggctcccaaagagtgcagaaaggctcgcttgaggatcgagatcaatggccaagactcgatatccgcgcatagcgaggtactgcgccagatgcgcggcggtggtggtcttacccgacccacctttgaaattcatcacagagataacctgaagctgctcgccgcctcgacgatgtggcaggtagcgccggttcccgcggccgacctgatccatatacttccgaatcacatggatatcttcaattgagaacattcgcctgccacctgggctcatgctaacattcaactctggcatctcagacgcggtctgccgtaaatatgactcgccaacgccgagcagcttggacgcctccgatggcccgaatgttcgaatacccttctcggaatgcggcgggaaaaccttaagatgatgtgcttgaagttggctcgagagggcatcggcatgacgctccatcaaggccgtcaaccctacaactacaggcgctgcttttaggacagacttcgccatctcaaacccattccttgccagtggcgatatttttcgcgaaactggaaaagttccgccgctggcaattagcgccgattctgctgtttgggcaagagcttttaggttaacagaaggttaacgccctcaggtcgaaaaactccacccaactgttatttgtatttatttccaatgccttagagagattgccatttgaatatgttcatgtattgttttagtgataatcctacaatcgtaacccaaaaagaggtcgccctctgcgcgccgtcgtccaatataggcgaagtcacccttgcgactcaggcggattctaccttgtaggatcgagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctca

SEQ ID NO: 4 - DNA, 33 bp, Artificial Sequence
  primer Atpap1 U-XbaI
ccagtgtctagactatctttgttccatggaggg

SEQ ID NO: 5 - DNA, 33 bp, Artificial Sequence
  Atpap1 L-SacI
ccagtggagctccacaaacgcaaacaaatgttc

SEQ ID NO: 6 - DNA, 33 bp, Artificial Sequence
  GhRDL1p U-HindIII
ccagtgaagcttaattagttatgtttggtaaat

SEQ ID NO: 7 - DNA, 33 bp, Artificial Sequence
  primer GhRDL1p L-XbaI
ccagtgtctagactagaacaggagtgactaatt

SEQ ID NO: 8 - DNA, 33 bp, Artificial Sequence
  primer GhEXPAp U-HindIII
ccagtgaagctttttaagcaaaaaattaatagt

SEQ ID NO: 9 - DNA, 33 bp, Artificial Sequence; identical to SEQ ID NO: 7
  primer GhEXPAp L-XbaI

SEQ ID NO: 10 - DNA, 33 bp, Artificial Sequence
  primer GhHOX3 U-XbaI
ccagtgtctagaatggattgcggaagcggcggc

SEQ ID NO: 11 - DNA, 33 bp, Artificial Sequence
  primer GhHOX L-SacI
ccagtggagctctcaagaactaggacaattcaa

SEQ ID NO: 12 - DNA, 33 bp, Artificial Sequence
  primer hspT L-PstI
actactctgcagaattccttatctttaatcata

SEQ ID NO: 13 - DNA, 33 bp, Artificial Sequence
  primer GhRDL1p U-SphI
ccagtggcatgcaattagttatgtttggtaaat

SEQ ID NO: 14 - DNA, 302 bp, Gossypium hirsutum
aattagttatgtttggtaaatgaatttgaatacatgcaccaccactctcaatgctcacccacctaagcccatgacataaactgcatttaagtgaaccagtggcagttggagccattatgttggttgaaaaatattgttggcagtgttattcagcagtacttgttctaaggctcctactacatttctcattataaatgtggagaaatcttgttctactttccattccatgcaacactaacttttagattccctttccattgcactgctctttctttctatagaattagtcactcctgttctag

SEQ ID NO: 15 - DNA, 2000 bp, Gossypium hirsutum
tttaagcaaaaaattaatagtaagttctatattagaaaattatttaatttcaatcaaacaatgcttaaataaattaaaaagttatataaaatataaaatttagacactataatattaaaataattcagacaactattatcatttttaataagttttctaaaataatgtatgcgaaaaacacttactttattaatacttaaataaatataatttattatatttacaaataaatatttgtatttattcatatgcaaatttttttaaaaatataaatattaaaaaaattatataatttcataaaagtggaaattaattattaaaaaatgaaaattaaataagattaattcattttcctaagaaatataaaaattctaggagctaacatatttaataatattatcatatttctaaaattagttaatattttaaattatggagactaaataagaaaattaaaaaagtgaaagcgctttataaaataaagtaaaaatattgaggggtttatgttacaaataacccaatcgcgtttcccatagagaaaaggaattttctttcttttattttcatgtcattttttcatacaatttctttggtcacttaaattctcaataaaattagaaaacagccctcaaaaataatattgatacgttgaaagatttatcaactttcgaccatcgacactttaaaaaaattagaaagttataatttttttttcaaaaaaactataatacctctctagttttagctaattaaattattattattttattattttattgttattaaaagtgtaacttgcactcaactattagtaagtttacgttttgatcacttaatttcagaaagttaaaaaatggtctttgaactattcgaaaattttcatttaagttactggaatatttaaaagtttttatttaagtcaccgggctattaagtttttttttaaaaattcgattagcaagttccaagctacgattcgataagtgatacaatggatttatacttattgacaaatagaatatacattaggtccaagttgatattacggtcagtgttgaaaatcgaaaaaaaatatttggattttgattcataaatttatgacttcaaagctggttcatgaaaaagaactaaagtgtaagagggaaggaaaaaaatatcttttgattggcacaaacagtgcgaacaaagaagaccacacaataacaattttaacaatatactaatttaaatgaaaaattttcaataatttaataagttaaccgaggaaaacttactaagagttagttacccctgttaaaataactttcatgaagtaatagaaacttttagtacgtatcatcttatatagaacaatttctattttcagaaagtcaagaaaattgtattctagaaaatggcgacttcttcaccttcagtccttccctgatcggcgcttgtgaaaaacgaaaaacctgagtctgattggctgactgaaaatgaacctactcatcaccattcactattaccaacttcaaatgataggggaattaactggtaaagtgtaactccaccgatggttgaggtggttggctggagttaaatgagatttttttagttttgtttcaagtggcttcaattgcaagcaattaggagactgcgctggaataacccctcgctcaaccttccgccattgttatggtttaattaaacattatgtttccatccatctatatttatatccattaaaacaagtcgttgagcaaataatggatactggataccatcatatctatgattaaaattttgcatgtgcccttttaatgtatagcttaagccttaattatcctccaaatttgtactctttcaccacttaattagctacgtacggtacttagcgttgcttgtcatcttctgtactacaaactctttctcattttgtataaatagctatacactttttctctcctcaaatcaataaggttaggtcagccaattgtttgagctagctagctcttactcaa

SEQ ID NO: 16 - DNA, 747 bp, Arabidopsis thaliana
atggagggttcgtccaaagggctgcgaaaaggtgcttggactactgaagaagatagtctcttgagacagtgcattaataagtatggagaaggcaaatggcaccaagttcctgtaagagctgggctaaaccggtgcaggaaaagttgtagattaagatggttgaactatttgaagccaagtatcaagagaggaaaacttagctctgatgaagtcgatcttcttcttcgccttcataggcttctagggaataggtggtctttaattgctggaagattacctggtcggaccgcaaatgacgtcaagaattactggaacactcatctgagtaagaaacatgaaccgtgttgtaagataaagatgaaaaagagagacattacgcccattcctacaacaccggcactaaaaaacaatgtttataagcctcgacctcgatccttcacagttaacaacgactgcaaccatctcaatgccccaccaaaagttgacgttaatcctccatgccttggacttaacatcaataatgtttgtgacaatagtatcatatacaacaaagataagaagaaagaccaactagtgaataatttgattgatggagataatatgtggttagagaaattcctagaggaaagccaagaggtagatattttggttcctgaagcgacgacaacagaaaagggggacaccttggcttttgacgttgatcaactttggagtcttttcgatggagagactgtgaaatttgattag

SEQ ID NO: 17 - DNA, 2142 bp, Gossypium hirsutum
atggattgcggaagcggcggcggcggcttaggagcgagtggttccggcggagaccatgattcctccgacctttcacgacggaaaaagccttaccatcgtcacacagctcaccagattcagaggcttgaatcgatgttcaaggaatgtccacacccagacgagaaacaaaggttacagctaagtagggaattgggattagcaccaagacagatcaagttttggtttcaaaataggaggacacaaatgaaggcacaacatgaaagagccgataactctgcacttcgtgctgagaacgataagatccgatgtgaaaacatagccataagagaagcacttaagaatgtgatttgtccttcttgtggtggtcctcctgctaatgaggattcttattttgatgaccagaaaatgagaatggagaatgctcaattaaaggaagagcttgatagagtatccagcattgcagccaagtacatagggagaccaatatcccaactcccaccagtacaacctgttcatatctcttcattagatttcaggatggcgagttttgacggctatggtgtcggcgctggtccttcactcgatctcgatctcctccccggaagttcgtcatcaatgccgaatttaccgttccaaccggtcgttatttcagacattgacaagtccctcatgtctgatatagccgcaaatgcaatggaagaactgcttaggctattacaaaccaatgaaccattgtggattaaatccactaatgatggcaaagatgctcttaatctcgaaagttatgaaagaatcttccctaagcctaataatactcattttaaatctcctaatattagggtcgaagcttctcgagattccggtgttgttattatgaatggcttagctttggttgacatgttcatggattccaacaaatggttggagctatttcccaccattgtgtcaattgccaaaacaatcgaagtgatttcacctgggatgttgggtacacatagtggttctttgcaattgatgtatgaagaactacaggtcttgtctccattagtaccaacacgtgaattttatacacttcgatattgtcaacaaattgaacaagggttatgggctattgtcaatgtttcatatgatttgccacaatttgcttctcaatgtcgatctcatagactcccttctggttgtttgattcaagacatgcctaatggttactccaaggttacttggttagaacatgtggagattgaagacaaaactccgattcatcgattgtatcgagaccttgttcatagtggttcagcatttggagccgaacggtggctcacgacccttcaaaggatgtgcgaacggtttgcttgcttaatggtatcaagcacttcgactcgggatctcggtggagtgattccatctcccgatggcaggagaagcatgatgaagctagctcaaaggatggtaaacaatttctgcacgagcgtcggcacgtcgaatagtcatcggtcgacaacactttccggttcgaatgaaattggtgttcgggtcaccgttcataagagttctgatcccggacaacccaacggtatagttcttagtgcagctactacattttggctccctgttagtccacaaaatgtcttcaatttcttcaaagatgaaagaactagaccacagtgggatgttctatccaacggcaacgcagtccaagaagttgctcatatcgcaaacggatcacatcccggcaactgtatatccgttttacgagcctttaatacaagtcacaacaacatgttaatactacaagagagttgcattgacagttctggttcactggtggtatactgtccagttgatttaccagctattaatgtagcaatgagcggcgaagatccatcttacattccgttacttccgtcaggttttacgataacacccgacggtcatctcgagcaaggtgacggagcatcgacgagttctagtactgggcacggaagatcgtccggtggttcattgattacggtagcgtttcaaatactagtgagcagcttgccatcggcaaaattaaacttggattcggtcacaattgttaataaccttattgctaatactgtacaacaaattaaggctgctttgaattgtcctagttcttga